(19) United States
(12) Patent Application Publication
(10) Pub. No.: US 2011/0130375 A1
(43) Pub. Date: Jun. 2, 2011

(54) NEW NO-RELEASING STEROIDS FOR THE TREATMENT OF RETINA AND MACULA LUTEA DISEASES

(75) Inventors: Francesca Benedini, San Donato Milanese (Milano) (IT); Rebecca Steele, Solaro(Milano) (IT); Valerio Chiroli, Milano (IT); Stefano Biondi, Pero(Milano) (IT); Ennio Ongini, Segrate(Milano) (IT)

(73) Assignee: NICOX S.A., Sophia Antipolis - Valbonne (FR)

(21) Appl. No.: 13/056,946

(22) PCT Filed: Jul. 24, 2009
(86) PCT No.: PCT/EP2009/059543

§ 371 Date: Jan. 31, 2011

Related U.S. Application Data
(60) Provisional application No. 61/086,375, filed on Aug. 5, 2008.

(51) Int. Cl. A61K 31/573; A61P 29/00; A61P 27/02; C07J 5/00

(52) U.S. Cl. 514/180; 552/574

(57) ABSTRACT

The invention relates to compounds of general formula (I) and pharmaceutically acceptable salts or stereoisomers thereof

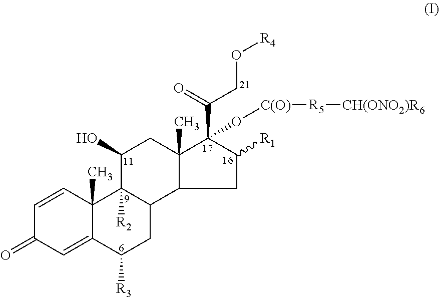

The compounds are useful for treating diabetic macular edema, diabetic retinopathy, macular degeneration, age-related macular degeneration and other diseases of retina and macula lutea.

The invention relates to nitrooxy derivatives of steroids, methods for their preparation, pharmaceutical compositions containing these compounds, and methods of using these compounds and compositions for treating ocular diseases, in particular diabetic macular edema, diabetic retinopathy, macular degeneration, age-related macular degeneration and other diseases of retina and macula lutea.

The retina is the part of the eye that is sensitive to light. The macula lutea is the region of the retina that allows us to read and recognize faces. Diseases of the macula, such as age-related macular degeneration and diabetic macular edema, account for major causes of blindness.

To combat these diseases, a variety of drugs have been investigated for their effects on blinding disorders. Currently, these drugs are delivered to the macula and retina via surgical procedures such as intravitreal or periorbital injections, or via systemic routes. Surgical methods often require repeated injections and may lead to serious ocular complications, including endophthalmitis, retinal detachment, and vitreous hemorrhage. Likewise, systemic administration is associated with a variety of potential systemic side effects and with the difficulty of delivering therapeutic levels of the drugs to the retina. Recently, there have been many reports of the effectiveness of intravitreal triamcinolone acetonide for the treatment of diffuse macular edema, refractory to laser treatment. Intravitreal triamcinolone injections are however associated with many ocular complications. The complications of intravitreal triamcinolone therapy include steroid induced elevation of intraocular pressure, cataractogenesis, post-operative infectious and non-infectious endophthalmitis, and pseudo-endophthalmitis.

At present chemotherapy, steroids and carbonic anhydrase inhibitors as major efficacy are used in symptomatic therapy, but their effectiveness is not established and their administration for a long time leads to occurrence of side effects such as cataract, steroid induced elevation of intraocular pressure, glaucoma, and infections thus continuous use of these drugs in chronic diseases, such as diabetes mellitus, is difficult under the circumstances.

EP 0929565 discloses compounds of general formula B—X₁—NO₂ wherein B contains a steroid residue, in particular hydrocortisone, and X₁ is a bivalent connecting bridge which is a benzyl ring, an alkyl chain or an ether. The compounds may be used in the treatment of ocular disorders.

EP 1 475 386 discloses compounds of formula A-B—C—NO₂ wherein A contains a steroid residue and B—C is a bivalent connecting bridge which contains an antioxidant residue. The compounds may be used in the treatment of oxidative stress and/or endothelial dysfunctions.

In the disclosed compounds the antioxidant linker is linked to the 21-OH of the steroid through a carboxylic group forming an ester bond.

WO 03/64443 discloses compounds of general formula B—X₁—NO₂ wherein B contains a steroid residue and X₁ is a bivalent connecting bridge which is a benzyl ring or a heterocyclic linker. The compounds may be used in the treatment of ocular diseases.

WO 07/025,632 discloses compounds of formula R—Z—X—ONO₂ wherein R—X contains triamcinolone acetonide, betamethasone valerate or prednisolone ethylcarbonate residue and X₁ is a bivalent connecting bridge which is an aromatic ring, an alkyl chain, an ether, ferulic acid, vanillic acid or an heterocyclic ring. The compounds may be used in the treatment of skin or mucosal membrane diseases and in particular in the treatment of atopic dermatitis, contact dermatitis and psoriasis.

F. Galassi et al. Br J Ophthalmology 2006, 90, 1414-1419 discloses the effects of an dexamethasone 21-[(4-nitrooxymethyl)]benzoate in a model of experimental corticosteroid-induced glaucoma in the rabbit. The NO-releasing dexamethasone was administered topically twice a day, the results show that the compound may prevent the intraocular pressure increase, the impairment of retro bulbar circulation, and the morphological changes in the ciliary bodies possibly induced by topical treatment with corticosteroids.

EP 1336602 discloses in general nitrate prodrugs, which include nitrate prodrugs of steroids, and their use for the prevention and the treatment of inflammatory, ischemic, degenerative and proliferative diseases of musculoskeletal, tegumental, respiratory, gastrointestinal, genito-urinary and central nervous systems. EP 1336602 shows that the absorption of these compounds by passive diffusion through biological membranes is slower than that of the known nitrate vasodilators.

WO 97/34871 discloses nitrosated or nitrosylated steroids having at least a nitroso and/or a nitrate group linked directly or indirectly to the positions 11 and/or 21 of the steroid moiety. The compounds can be used for the prophylaxis or the treatment of respiratory disorders.

It is an object of the present invention to provide nitrooxy-derivatives of steroids for treating inflammatory diseases.

Another object of the present invention to provide nitrooxy-derivatives of steroids for the prevention or the treatment of ocular diseases, in particular diabetic macular degeneration, diabetic retinopathy, age-related macular degeneration and other diseases of retina and macula lutea. In one aspect of the invention, one or more of these compounds reduce the side effects associated with the standard therapy with steroids. In a further embodiment, one or more of these compounds possess improved pharmacological activity compared to current standard therapy.

An object of the present invention is a compound of general formula (I) and pharmaceutically acceptable salts or stereoisomers thereof

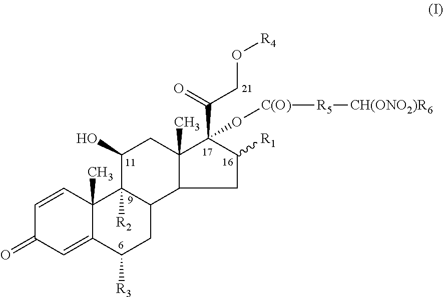

wherein
R₁ is CH₃ or OH, R₂ is F or Cl and R₃ is H or F, with the proviso that:

when R₁ is CH₃ then R₃ is H,

when R₁ is OH then R₂ is F;

when R₁ is CH₃, the CH₃ is linked to the carbon atom 16 in β position,
when R₁ is OH, the OH is linked to the carbon atom 16 in α position;
R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;
R₄ is —H or R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

### (F) —(Z)—Y

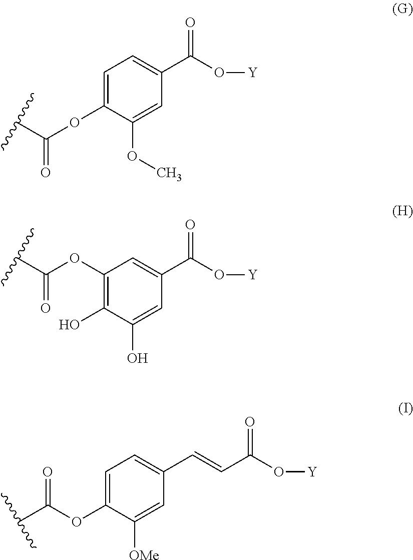

wherein:
R¹ is selected from:

### R^{1a})

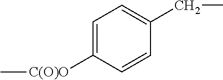

### —C(O)—S—CH₂—, —C(O)O—CH(CH₃)—, —C(O)O—CH₂—;

preferably R^{1a} is

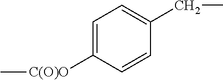

### R^{1b})

—C(O)—CH₂—, —C(O)—(CH₂)₂—; preferably R^{1b} is —C(O)—CH₂—;

### R² is —H or —C(O)CH₃;

R³ is —H, —CH₃, isopropyl, isobutyl, sec-butyl, methylthio-(CH₂)₂—, benzyl; preferably R³ is H or —CH₃;
R⁴ is —H, —CH₃, isopropyl, isobutyl, sec-butyl, methylthio-(CH₂)₂—, benzyl; preferably R⁴ is H or —CH₃;
Z is —C(O) or —C(O)—X″, wherein X″ is O, S or NR₁₂ wherein R₁₂ is H or a C₁-C₄ alkyl; preferably X″ is O;
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl; preferably R₈ is H or —CH₃;
R₉ and R₁₀ at each occurrence are independently H or a straight or branched C₁-C₁₀ alkylene, preferably R₉ and R₁₀ are H or —CH₃;
o and r are integers from 1 to 6; preferably o and r are integers from 1 to 4, more preferably o is 1 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O;
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4;
q is from 0 to 4,
t is 0 or 1,

### X is O.

Another embodiment of the present invention provides a compound of formula (I) above reported wherein

R₁ is CH₃ linked to carbon atom 16 in β position,

### R₂ is F and R₃ is H,

R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;
R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

### (F) —(Z)—Y

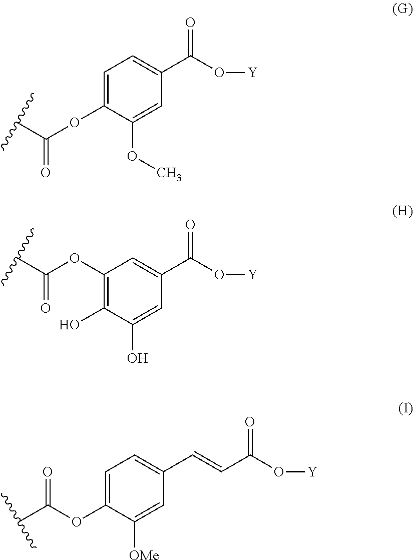

wherein:
R¹ is selected from:

### R^{1a})

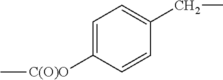

### —C(O)—S—CH₂—, —C(O)O—CH(CH₃)—, C(O)O—CH₂—;

preferably R^{1a} is

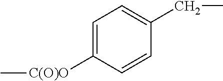

### R^{1b})

—C(O)—CH₂—, —C(O)—(CH₂)₂—; preferably R^{1b} is —C(O)—CH₂—;

### R² is —H or —C(O)CH₃;

R³ is —H, —CH₃, isopropyl, isobutyl, sec-butyl, methylthio-(CH₂)₂—, benzyl; preferably R³ is H or —CH₃;
R⁴ is —H, —CH₃, isopropyl, isobutyl, sec-butyl, methylthio-(CH₂)₂—, benzyl; preferably R⁴ is H or —CH₃;
Z is —C(O) or —C(O)—X″, wherein X″ is O, S or NR₁₂ wherein R₁₂ is H or a C₁-C₄ alkyl; preferably X″ is O;
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]ₚ—[(CH₂)ᵣ—X]ₛ—(CH₂)_{q}—CH(ONO₂) —(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl; preferably R₈ is H or —CH₃;
R₉ and R₁₀ at each occurrence are independently H or a straight or branched C₁-C₁₀ alkylene; preferably R₉ and R₁₀ are H or —CH₃;
o an r are integers from 1 to 6; preferably o and r are integers from 1 to 4, more preferably o is 1 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O,
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4;
q is from 0 to 4,
t is 0 or 1,

### X is O.

Another embodiment of the present invention provides a compound of formula (I) above reported wherein

R₁ is OH linked to the carbon atom 16 in α position,

### R₂ is F and R₃ is F,

R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;
R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

### (F) —(Z)—Y

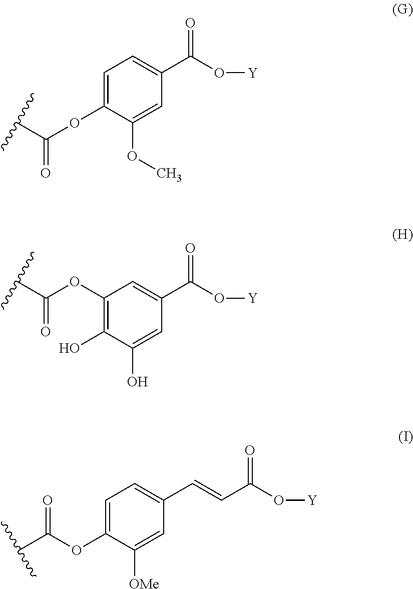

wherein:
R¹ is selected from:

### R^{1a})

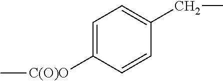

### —C(O)—S—CH₂—, —C(O)O—CH(CH₃)—, —C(O)O—CH₂—;

preferably R^{1a} is

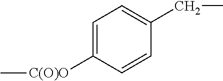

### R^{1b})

—C(O)—CH₂—, —C(O)—(CH₂)₂—; preferably R^{1b} is —C(O)—CH₂—;

### R² is —H or —C(O)CH₃;

R³ is —H, —CH₃, isopropyl, isobutyl, sec-butyl, methylthio-(CH₂)₂—, benzyl, preferably R³ is H or —CH₃;
R⁴ is —H, —CH₃, isopropyl, isobutyl, sec-butyl, methylthio-(CH₂)₂—, benzyl; preferably R⁴ is H or —CH₃;
Z is —C(O) or —C(O)—X″, wherein X″ is O, S or NR₁₂ wherein R₁₂ is H or a C₁-C₄ alkyl; preferably X″ is O;
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl, preferably R₈ is H or —CH₃;
R₉ and R₁₀ at each occurrence are independently H or a straight or branched C₁-C₁₀ alkylene, preferably R₉ and R₁₀ are H or —CH₃;
o and r are integers from 1 to 6; preferably o and r are integers from 1 to 4, more preferably o is 1 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁ₗ is H or a C₁-C₄ alkyl; preferably X is O
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4,
q is from 0 to 4,
t is 0 or 1,

### X is O.

Another embodiment of the present invention provides a compound of formula (I) above reported wherein

R₁ is CH₃ linked to the carbon atom 16 in β position;

### R₂ is Cl;

### R₃ is H;

R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;
R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

### (F) —(Z)—Y

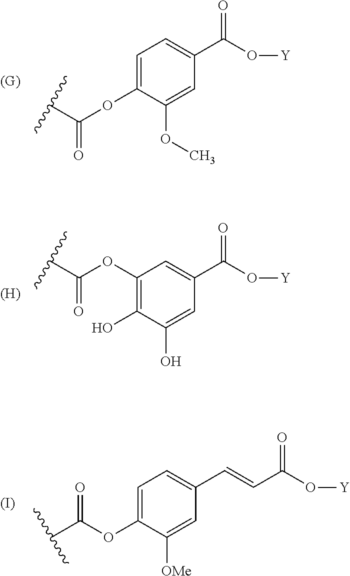

wherein:
R¹ is selected from:

### R^{1a})

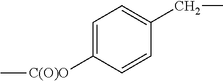

### —C(O)—S—CH₂—, —C(O)O—CH(CH₃)—, —C(O)O—CH₂—;

preferably R^{1a} is

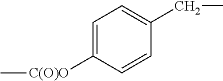

### R^{1b})

—C(O)—CH₂—, C(O)—(CH₂)₂—; preferably R^{1b} is —C(O)—CH₂—;

### R² is —H or —C(O)CH₃;

R³ is —H, —CH₃, isopropyl, isobutyl, sec-butyl, methylthio-(CH₂)₂—, benzyl; preferably R³ is H or —CH₃;
R⁴ is —H, —CH₃, isopropyl, isobutyl, sec-butyl, methylthio-(CH₂)₂—, benzyl; preferably R⁴ is H or —CH₃;
Z is —C(O) or —C(O)—X″, wherein X″ is O, S or NR₁₂ wherein R₁₂ is H or a C₁-C₄ alkyl; preferably X″ is O;
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl; preferably R₈ is H or —CH₃;
R₉ and R₁₀ at each occurrence are independently H or a straight or branched C₁-C₁₀ alkylene; preferably R₉ and R₁₀ are H or —CH₃;
o and r are integers from 1 to 6; preferably o and r are integers from 1 to 4, more preferably o is 1 or 2 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4,
q is from 0 to 4,
t is 0 or 1,

### X is O.

Another embodiment of the present invention provides a compound of formula (I) above reported wherein

R₁ is —OH linked to the carbon atom 16 in α position;

### R₂ is F,

### R₃ is H,

R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;
R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

### (F) —(Z)—Y

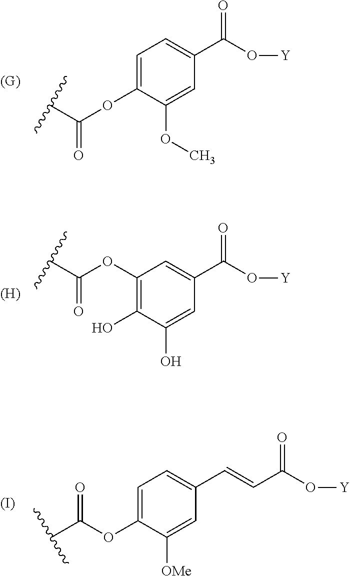

wherein:
R¹ is selected from:

### R^{1a})

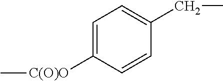

### —C(O)—S—CH₂—, —C(O)O—CH(CH₃)—, —C(O)O—CH₂—;

preferably R^{1a} is

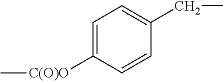

### R^{1b})

—C(O)—CH₂—, —C(O)—(CH₂)₂—; preferably R^{1b} is —C(O)—CH₂—;

### R² is —H or —C(O)CH₃;

R³ is —H, —CH₃, isopropyl, isobutyl, sec-butyl, methylthio-(CH₂)₂—, benzyl; preferably R³ is H or —CH₃;
R⁴ is —H, —CH₃, isopropyl, isobutyl, sec-butyl, methylthio-(CH₂)₂—, benzyl; preferably R⁴ is H or —CH₃;
Z is —C(O) or —C(O)—X″, wherein X″ is O, S or NR₁₂ wherein R₁₂ is H or a C₁-C₄ alkyl; preferably X″ is O;
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl; preferably R₈ is H or —CH₃;
R₉ and R₁₀ at each occurrence are independently H or a straight or branched C₁-C₁₀ alkylene; preferably R₉ and R₁₀ are H or —CH₃;
o and r are integers from 1 to 6; preferably o and r are integers from 1 to 4, more preferably o is 1 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O,
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4,
q is from 0 to 4,
t is 0 or 1,

### X is O.

Another embodiment of the present invention provides a compound of formula (I) above reported wherein

R₁ is CH₃ linked to the carbon atom 16 in β position,

### R₂ is F,

### R₃ is H,

### R₄ is —H,

R₅ is a straight or branched C₁-C₁₀ alkylene; preferably R₅ is a straight C₁-C₆ alkylene,
R₆ is H or a straight or branched C₁-C₄ alkyl; preferably R₆ is H or —CH₃, more preferably R₆ is H.

Another embodiment of the present invention provides a compound of formula (I) above reported wherein

R₁ is OH and it is linked to the carbon atom 16 in α position,

### R₂ is F,

### R₃ is F,

### R₄ is —H,

R₅ is a straight or branched C₁-C₁₀ alkylene; preferably R₅ is a straight C₁-C₆ alkylene,
R₆ is H or a straight or branched C₁-C₄ alkyl; preferably R₆ is H or —CH₃, more preferably R₆ is H.

Another embodiment of the present invention relates to compounds of formula (I) above reported wherein

R₁ is CH₃ linked to the carbon atom 16 in β position,

### R₂ is Cl,

### R₃ is H,

### R₄ is —H,

R₅ is a straight or branched C₁-C₁₀ alkylene; preferably R₅ is a straight C₁-C₆ alkylene,
R₆ is H or a straight or branched C₁-C₄ alkyl; preferably R₆ is H or —CH₃, more preferably R₆ is H.

Another embodiment of the present invention provides a compound of formula (I) above reported wherein

R₁ is OH linked to the carbon atom 16 of the steroidal in α position;

### R₂ is F,

### R₃ is H,

### R₄ is —H,

R₅ is a straight or branched C₁-C₁₀ alkylene; preferably R₅ is a straight C₁-C₆ alkylene,
R₆ is H or a straight or branched C₁-C₄ alkyl; preferably R₆ is H or —CH₃, more preferably R₆ is H.

Another embodiment of the invention provides a compound of general formula (I) and pharmaceutically acceptable salts or stereoisomers thereof

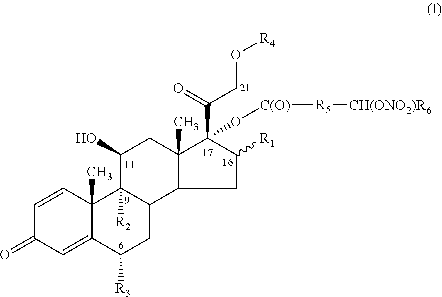

wherein
R₁ is CH₃ or OH, R₂ is F or Cl and R₃ is H or F, with the proviso that:

when R₁ is CH₃ then R₃ is H,

when R₁ is OH then R₂ is F;

when R₁ is CH₃, the CH₃ is linked to the carbon atom 16 of the steroidal structure in β position,
when R₁ is OH, the OH is linked to the carbon atom 16 of the steroidal structure in α position;
preferably in formula (I) R₁ is CH₃ linked to the carbon atom 16 in β position and R₂ is F or
R₁ is OH linked to the carbon atom 16 in α position and R₃ is F;
R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;

### R₄ is

### (F) —(Z)—Y

wherein
Z is —C(O) or —C(O)—X″, wherein X″ is O, S or NR₁₂ wherein R₁₂ is H or a C₁-C₄ alkyl; preferably X″ is O;
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl; preferably R₈ is H or —CH₃;
R₉ and R₁₀ at each occurrence are independently H or a straight or branched C₁-C₁₀ alkylene; preferably R₉ and R₁₀ are H or —CH₃;
o and r are integers from 1 to 6; preferably o and r are integers from 1 to 4, more preferably o is 1 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O,
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4,
q is from 0 to 4,
t is 0 or 1,

### X is O.

Another embodiment of the invention provides a compound of general formula (I) and pharmaceutically acceptable salts or stereoisomers thereof

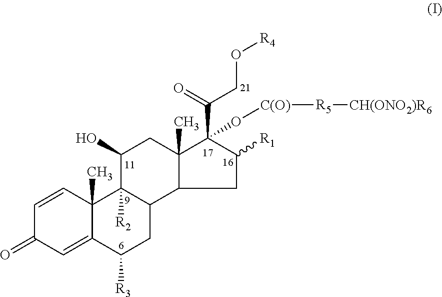

wherein
R₁ is CH₃ or OH, R₂ is F or Cl and R₃ is H or F, with the proviso that:

when R₁ is CH₃ then R₃ is H,

when R₁ is OH then R₂ is F;

when R₁ is CH₃, the CH₃ is linked to the carbon atom 16 of the steroidal structure in β position,
when R₁ is OH, the OH is linked to the carbon atom 16 of the steroidal structure in α position,
preferably in formula (I) R₁ is CH₃ linked to the carbon atom 16 in β position and R₂ is F or
R₁ is OH linked to the carbon atom 16 in α position and R₃ is F;
R₅ is a straight or branched C₁-C₁₀ alkylene; preferably R₅ is a straight C₁-C₆ alkylene;
R₆ is H or a straight or branched C₁-C₄ alkyl; preferably R₆ is H or —CH₃, more preferably R₆ is H;
R₄ is selected from:

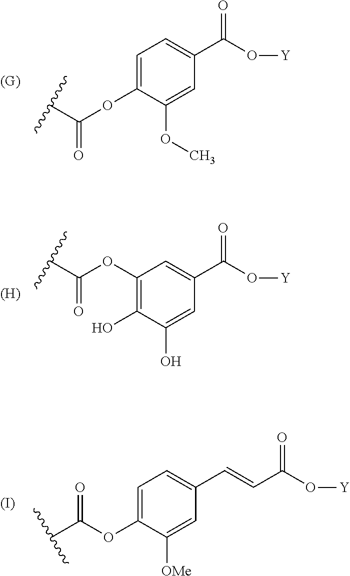

wherein:
Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl; preferably R₈ is H or —CH₃;
R₉ and R₁₀ at each occurrence are independently H or a straight or branched C₁-C₁₀ alkylene; preferably R₉ and R₁₀ are H or —CH₃;
o and r are integers from 1 to 6; preferably o and r are integers from 2 to 4, more preferably o and r are 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O,
preferably Y is selected from

### —R₇—CH(ONO₂)R₈

—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
o and r are integers from 1 to 4,
p and s are from 1 to 4,
q is from 0 to 4,
t is 0 or 1,

### X is O.

Another embodiment of the invention provides a compound of general formula (I) and pharmaceutically acceptable salts or stereoisomers thereof

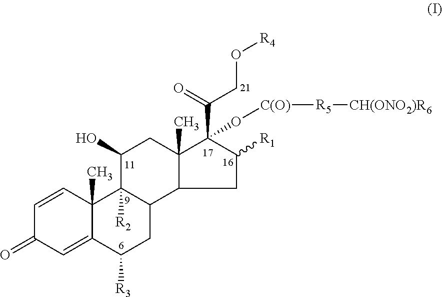

wherein
R₁ is CH₃ or OH, R₂ is F or Cl and R₃ is H or F, with the proviso that:

when R₁ is CH₃ then R₃ is H,

when R₁ is OH then R₂ is F;

when R₁ is CH₃, the CH₃ is linked to the carbon atom 16 of the steroidal structure in β position,
when R₁ is OH, the OH is linked to the carbon atom 16 of the steroidal structure in α position,
preferably in formula (I) R₁ is CH₃ linked to the carbon atom 16 in β position and R₂ is F or
R₁ is OH linked to the carbon atom 16 in α position and R₃ is F,
R₅ is a straight or branched C₁-C₁₀ alkylene; preferably R₅ is a straight C₁-C₆ alkylene;
R₆ is H or a straight or branched C₁-C₄ alkyl, preferably R₆ is H or —CH₃, more preferably R₆ is H;
R₄ is selected from:

### (A) —R¹—OH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

wherein:
R¹ is selected from:

### R^{1a})

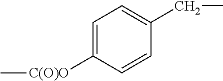

### —C(O)—S—CH₂—, —C(O)O—CH(CH₃)—, C(O)O—CH₂—;

preferably R^{1a} is

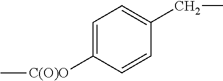

### R^{1b})

—C(O)—CH₂—, —C(O)—(CH₂)₂—; preferably R^{1b} is —C(O)—CH₂—;

### R² is —H or —C(O)CH₃;

R³ is —H, —CH₃, isopropyl, isobutyl, sec-butyl, methylthio-(CH₂)₂—, benzyl; preferably R³ is H or —CH₃;
R⁴ is —H, —CH₃, isopropyl, isobutyl, sec-butyl, methylthio-(CH₂)₂—, benzyl; preferably R⁴ is H or —CH₃;
Y is selected from

### —R₇—CH(ONO₂)R₈

—R₇—CH(ONO₂)—(CH₉R₁₀)ₜ—CH(ONO₂)R₈
—[(CH₂)ₒ—X]ₚ—[(CH₂)ᵣ—X]ₛ—(CH₂)—(CR₉R₁₀)ₜ—CH(ONO₂)R₈
—[(CH₂)ₒ—X]ₚ—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene; preferably R₇ is a straight C₁-C₆ alkylene;
R₈ is H or a straight or branched C₁-C₄ alkyl, preferably R₈ is H or —CH₃;
R₉ and R₁₀ at each occurrence are independently H or a straight or branched C₁-C₁₀ alkylene; preferably R₉ and R₁₀ are H or —CH₃;
o and r are integers from 1 to 6; preferably o and r are integers from 1 to 4, more preferably o is 1 or 2 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₁ wherein R₁₁ is H or a C₁-C₄ alkyl; preferably X is O,
preferably Y is selected from

### —R₇—CH(ONO₂)R₈

—R₇—CH(ONO₂)—(CR₉R₁₀)ₜ—CH(ONO₂)R₈
wherein
R₇ is a straight C₁-C₆ alkylene;

### R₈ is H or —CH₃;

R₉ and R₁₀ at each occurrence are independently H or —CH₃;
t is 0 or 1.

Another embodiment of the invention provides a compound selected from the group:

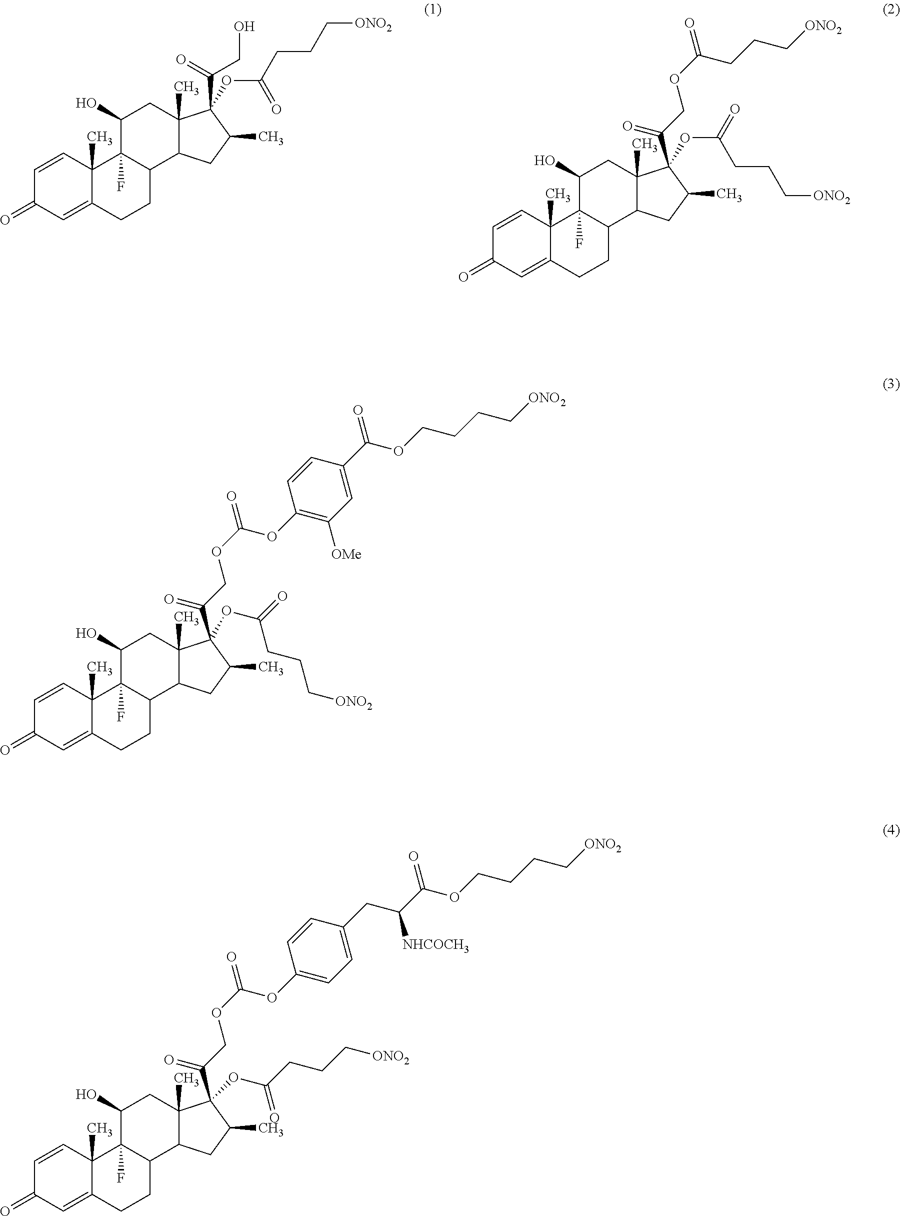

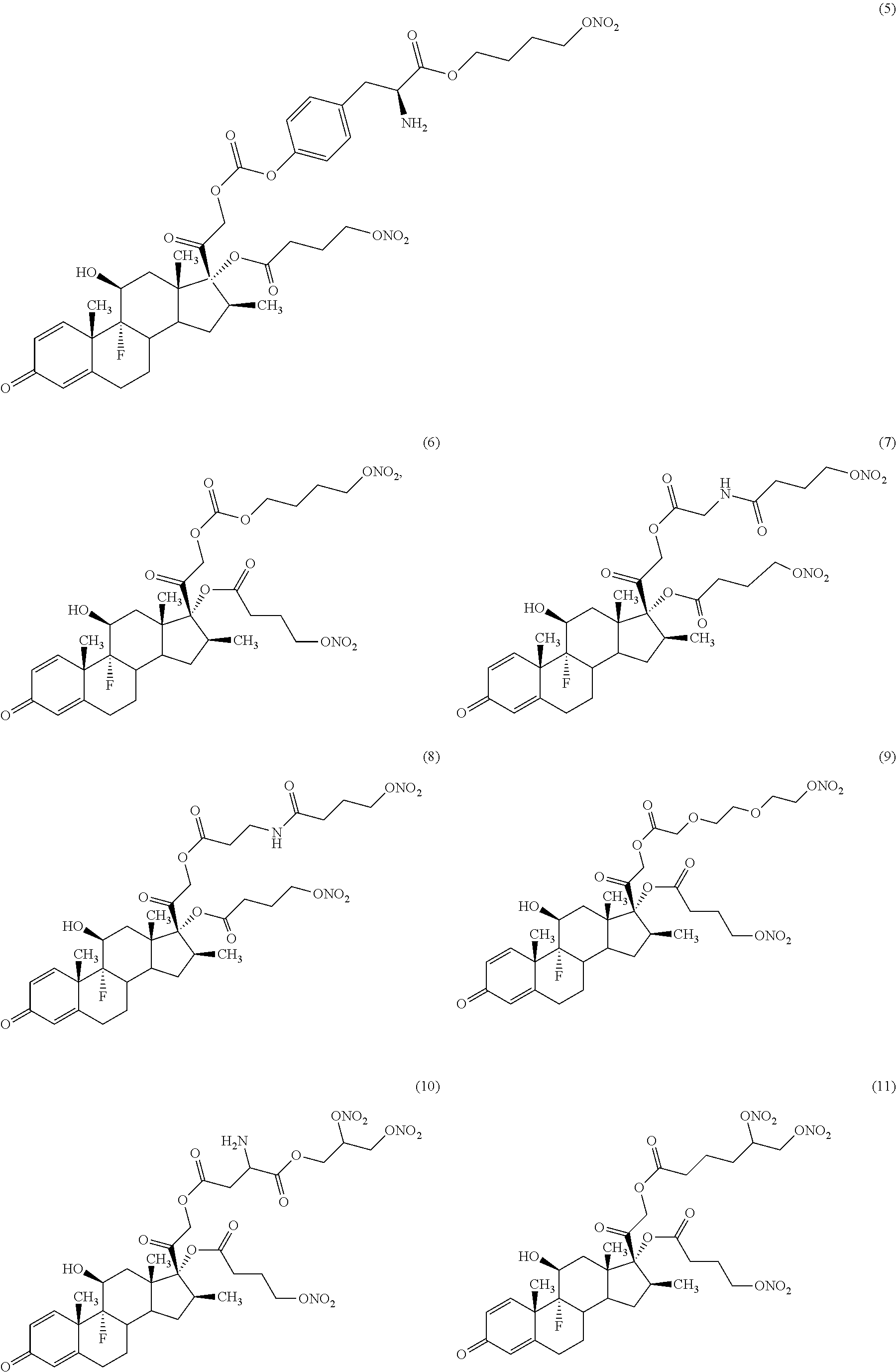

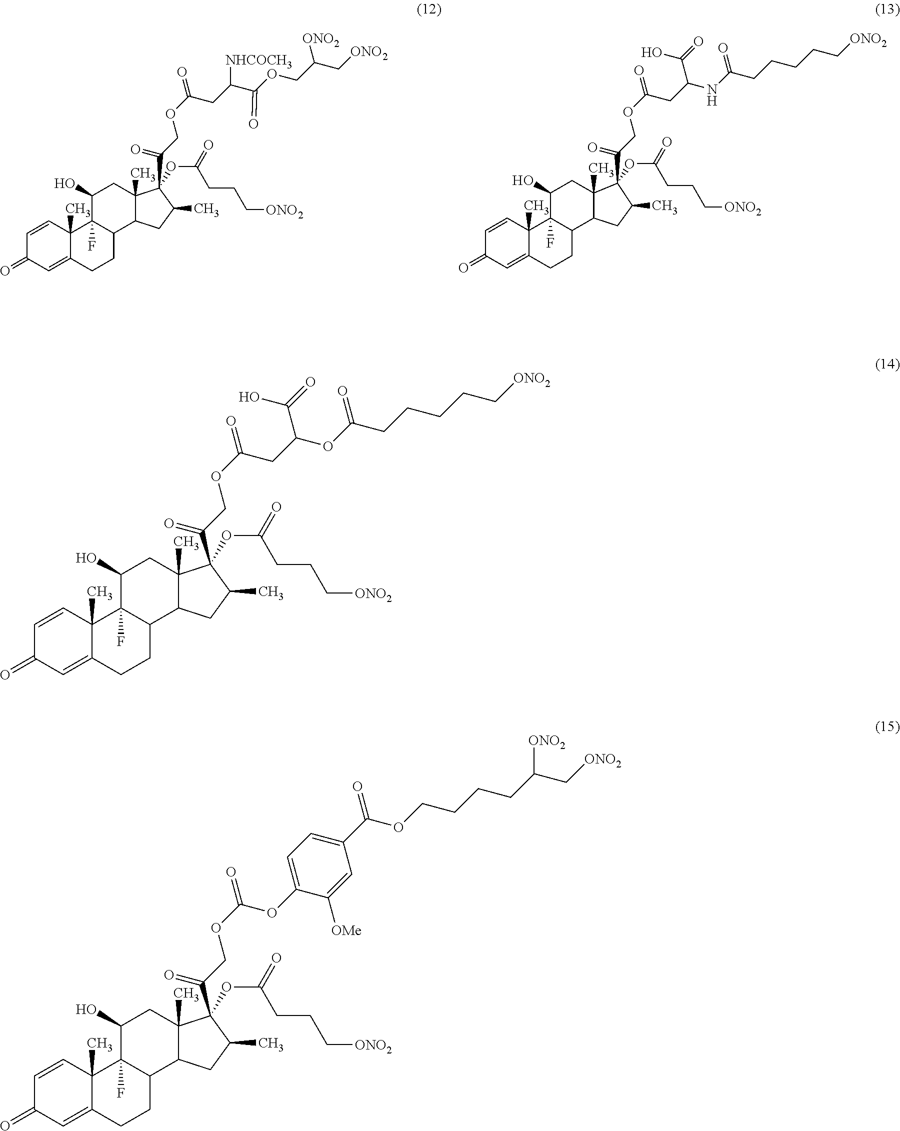

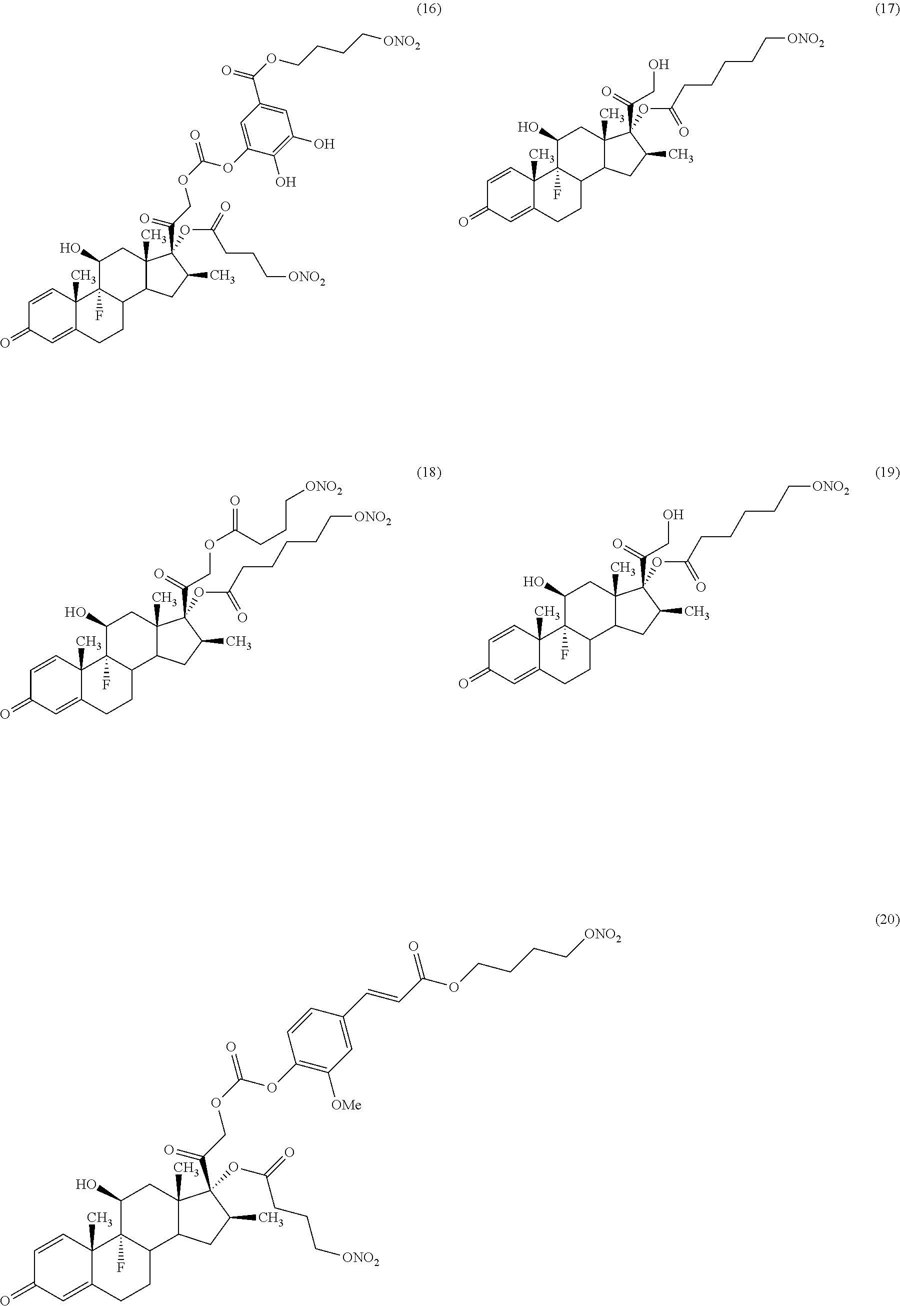

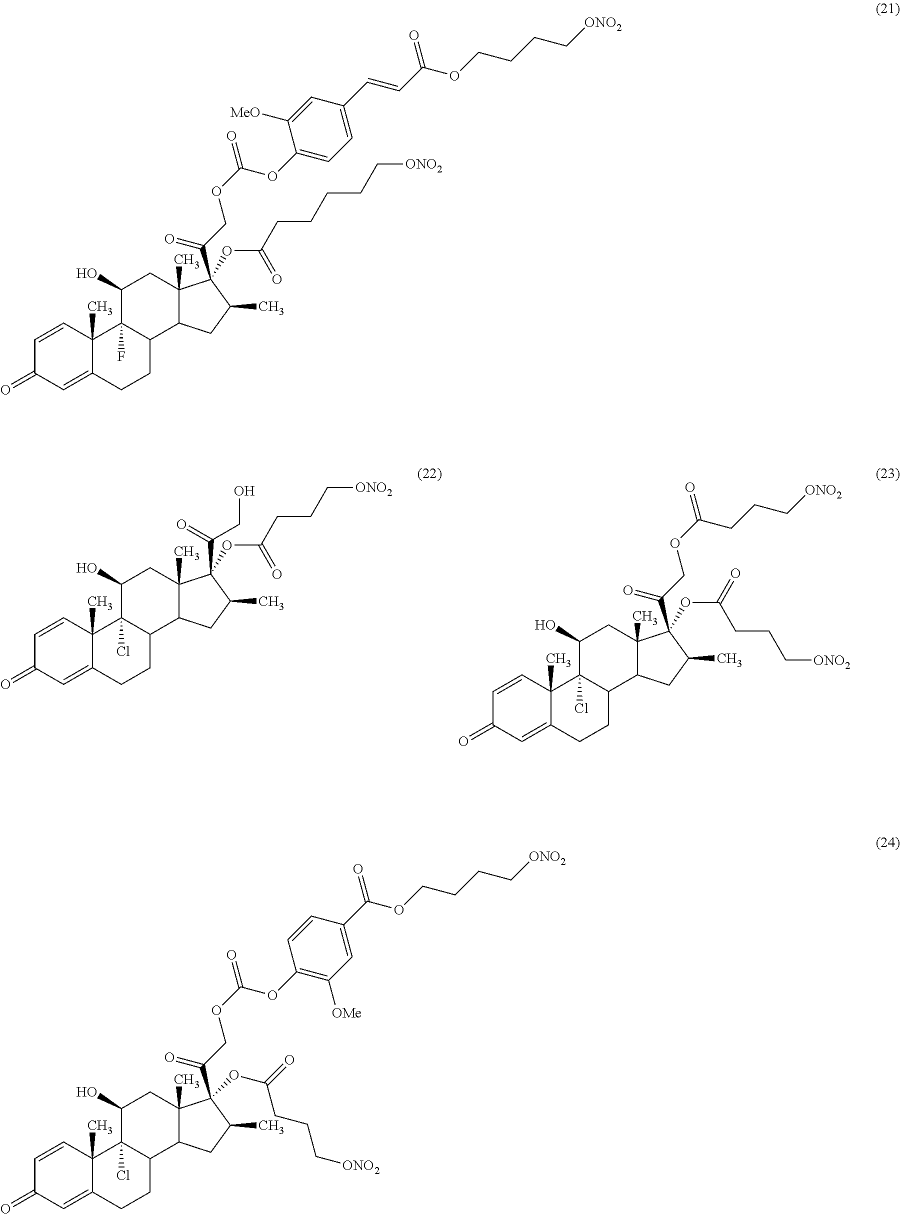

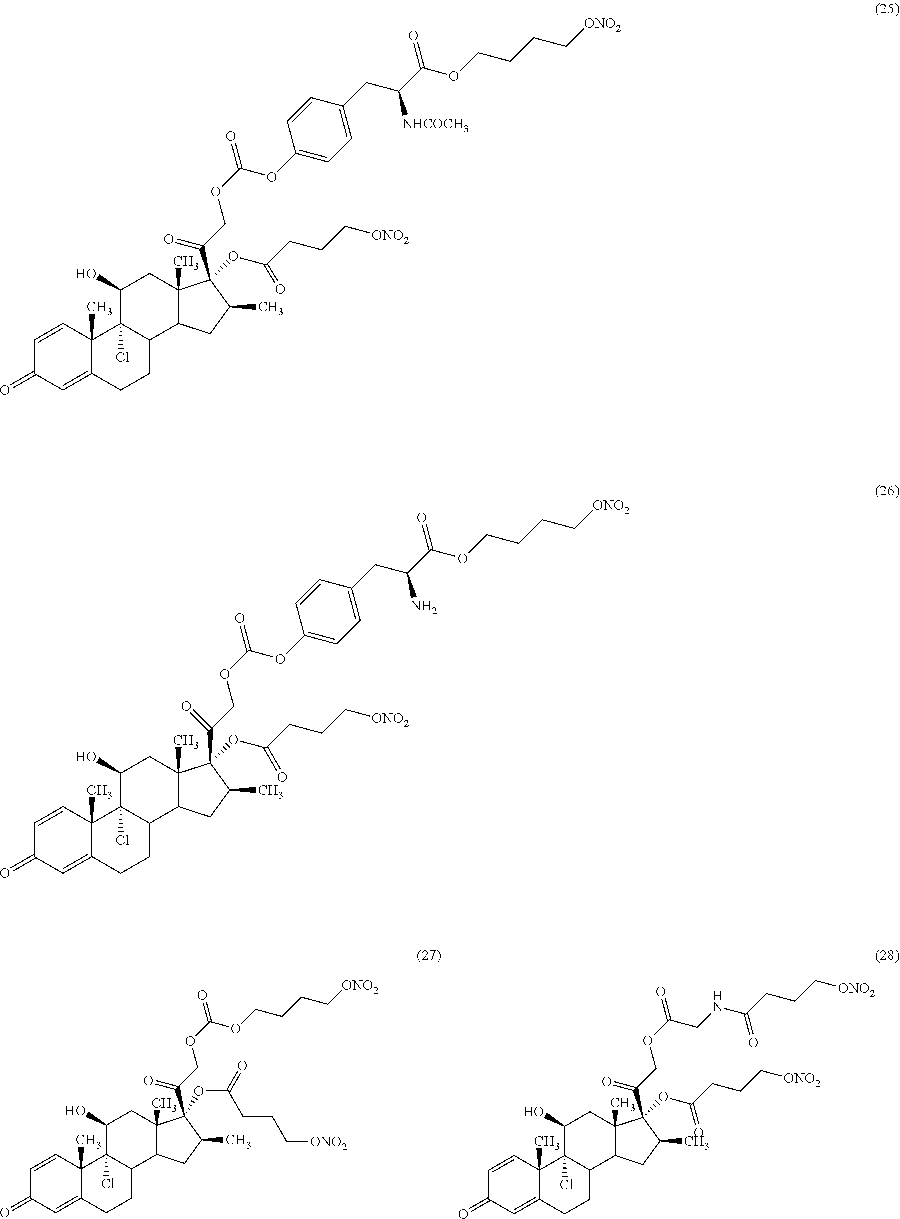

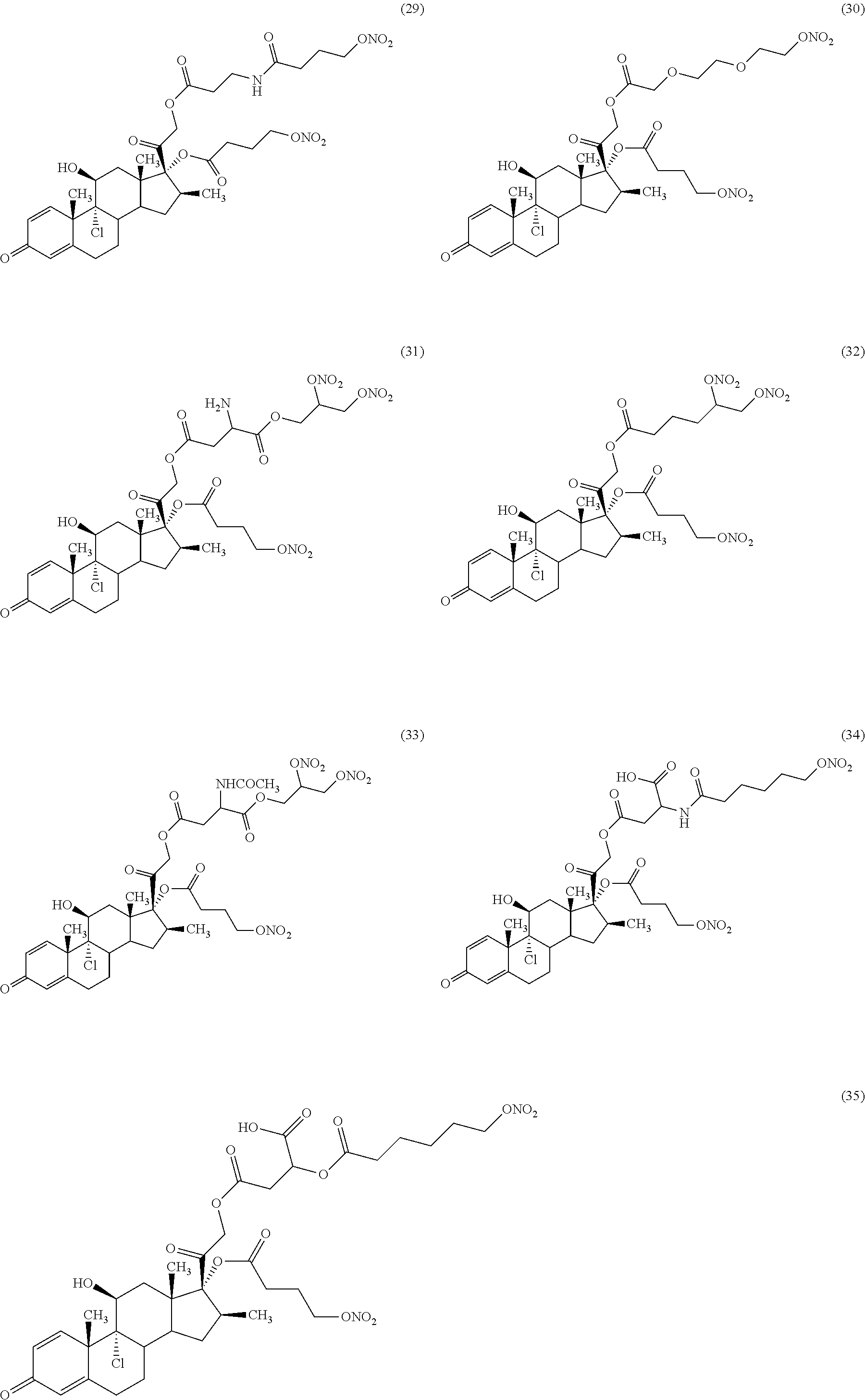

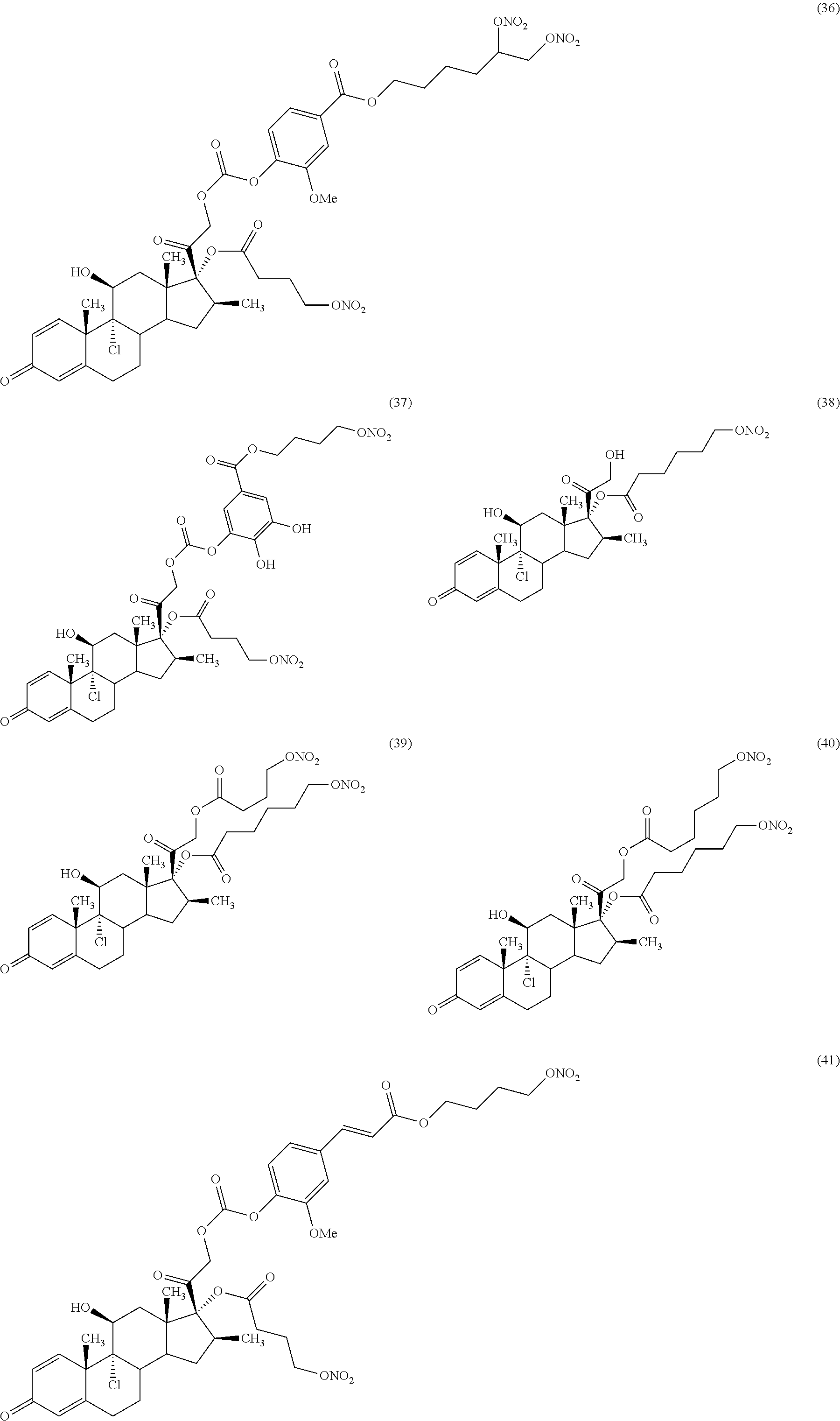

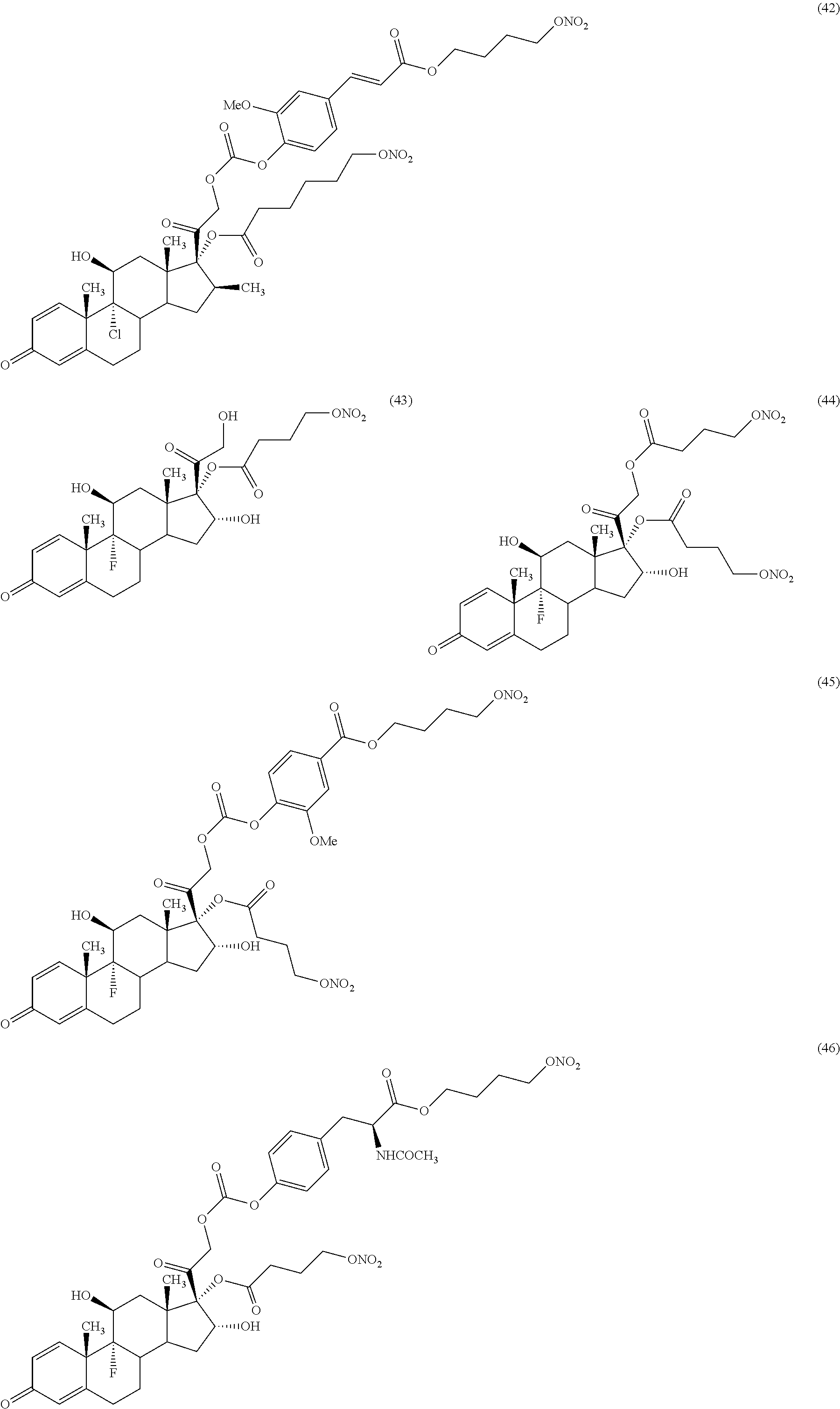

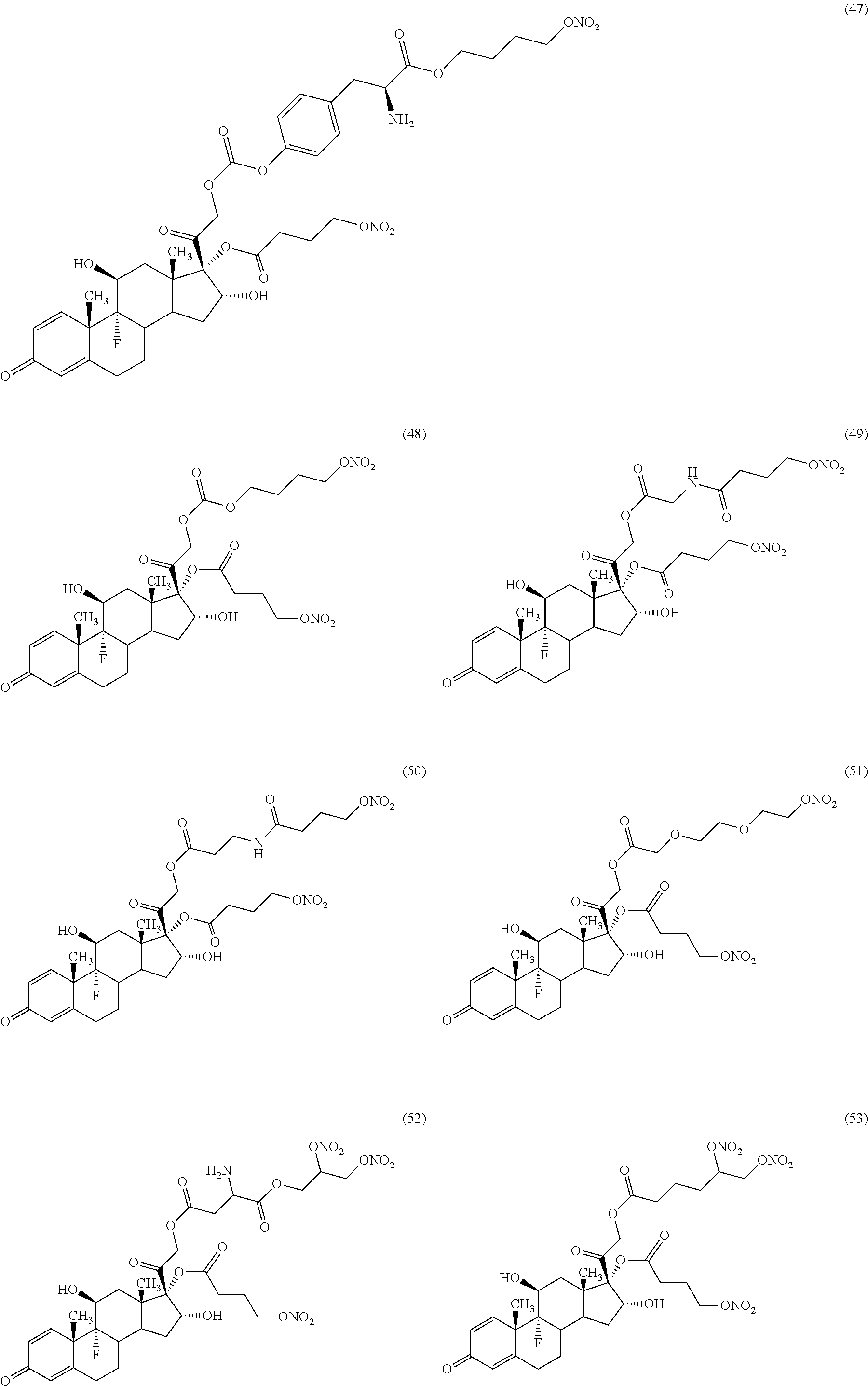

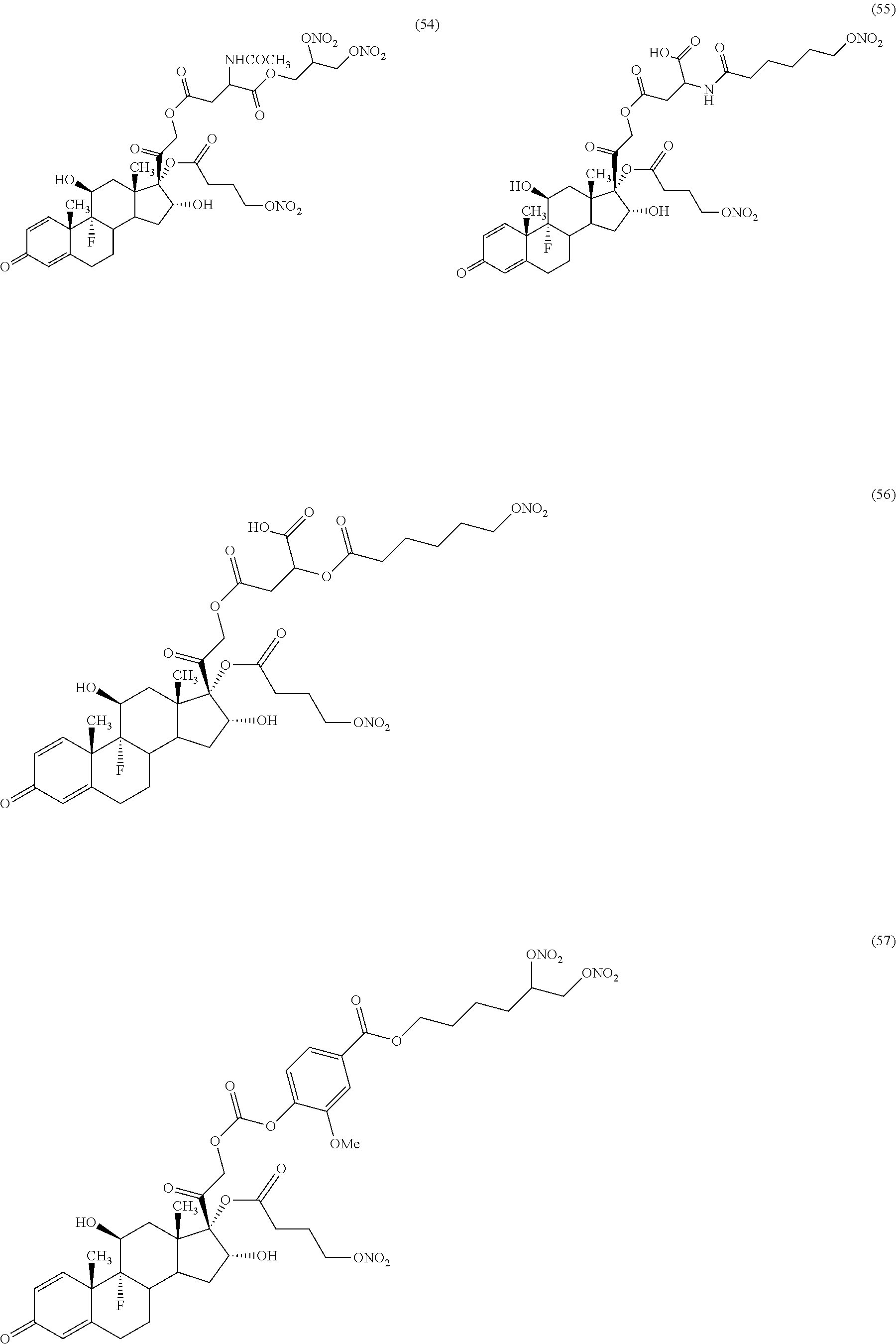

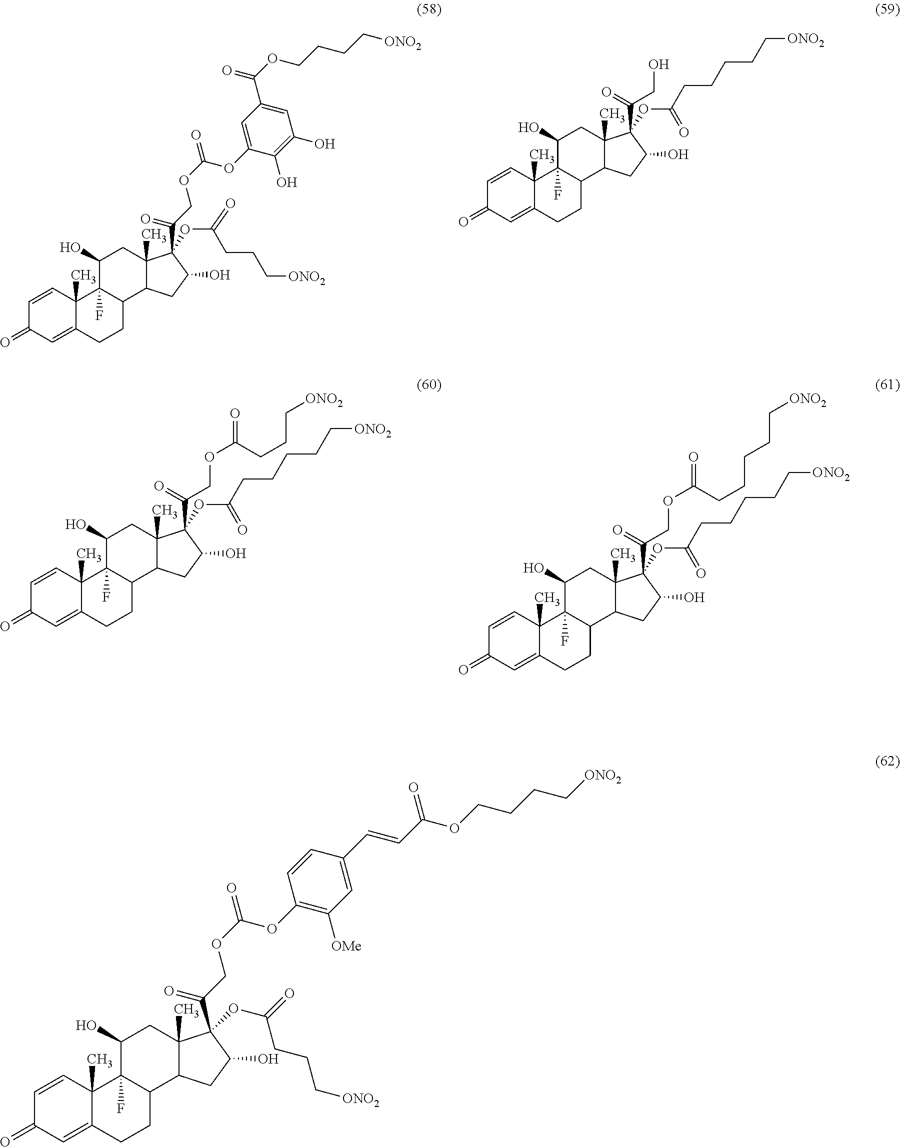

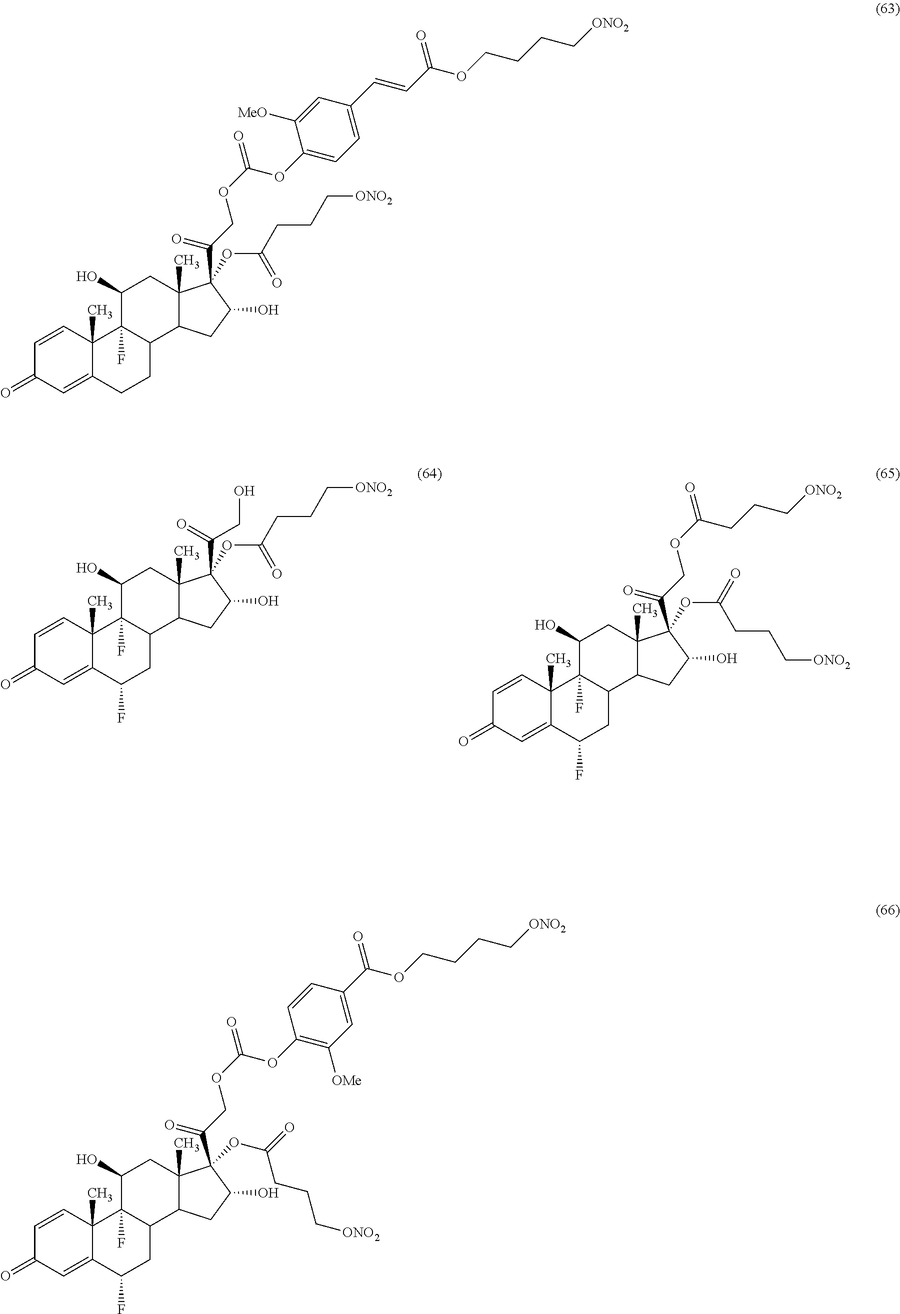

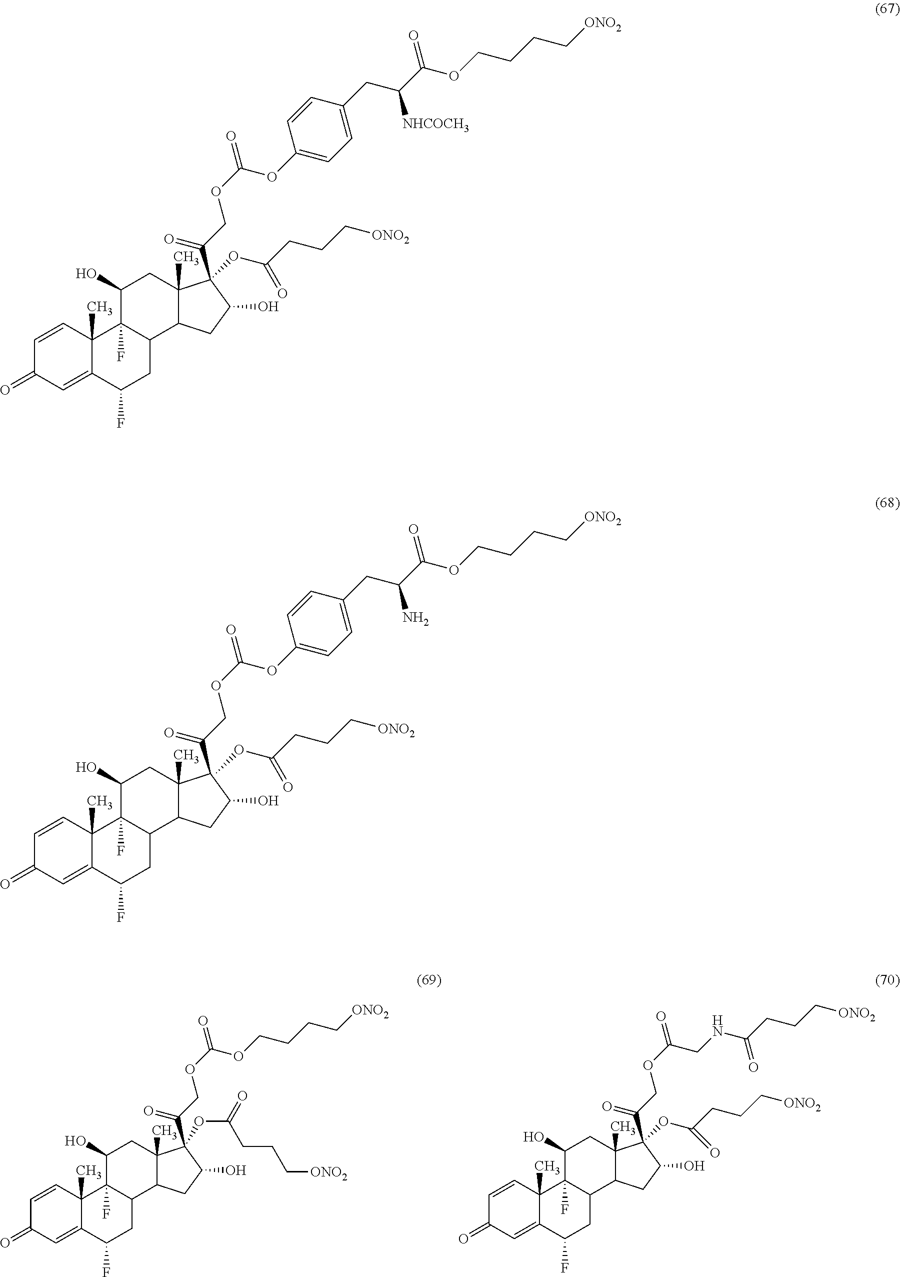

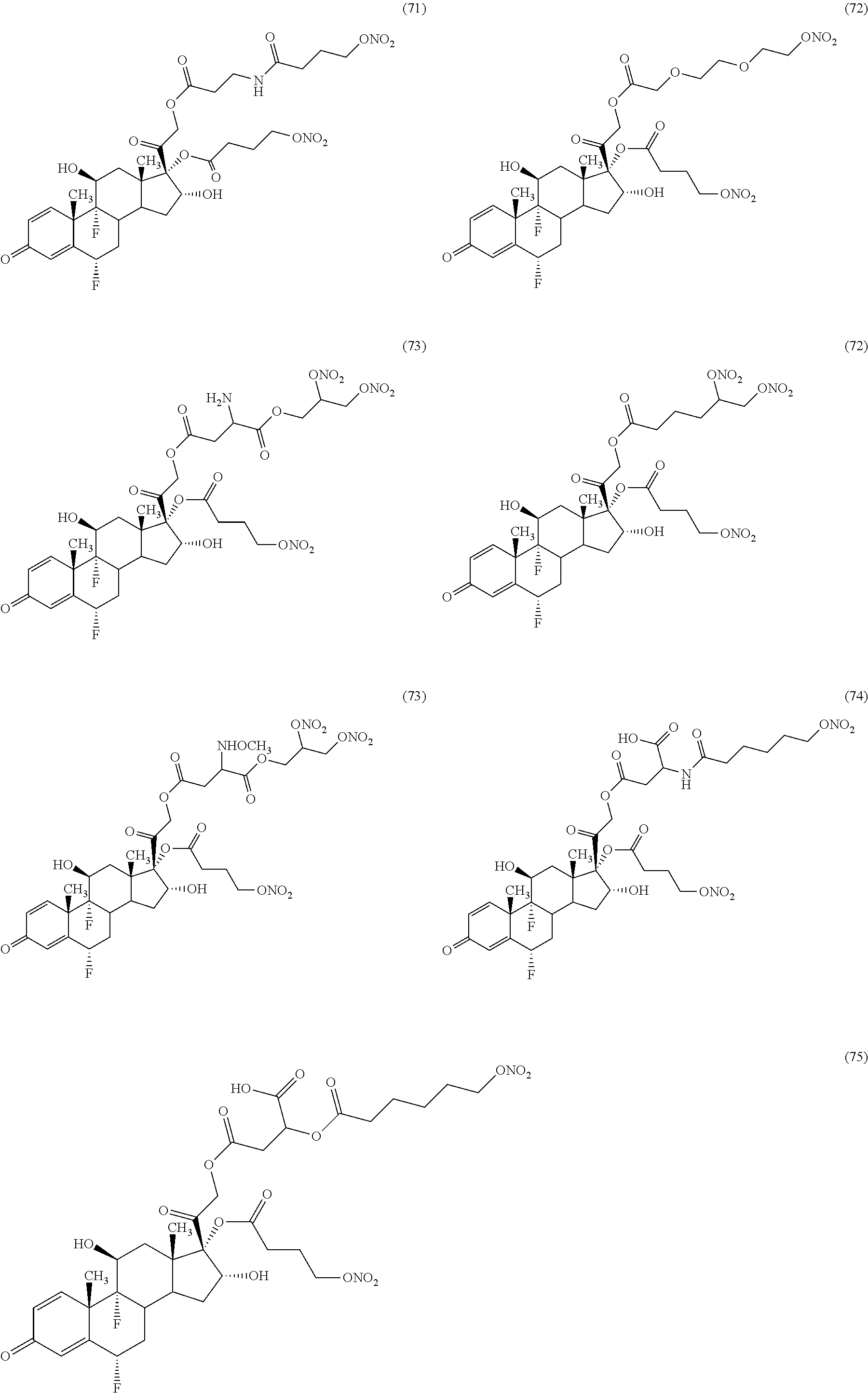

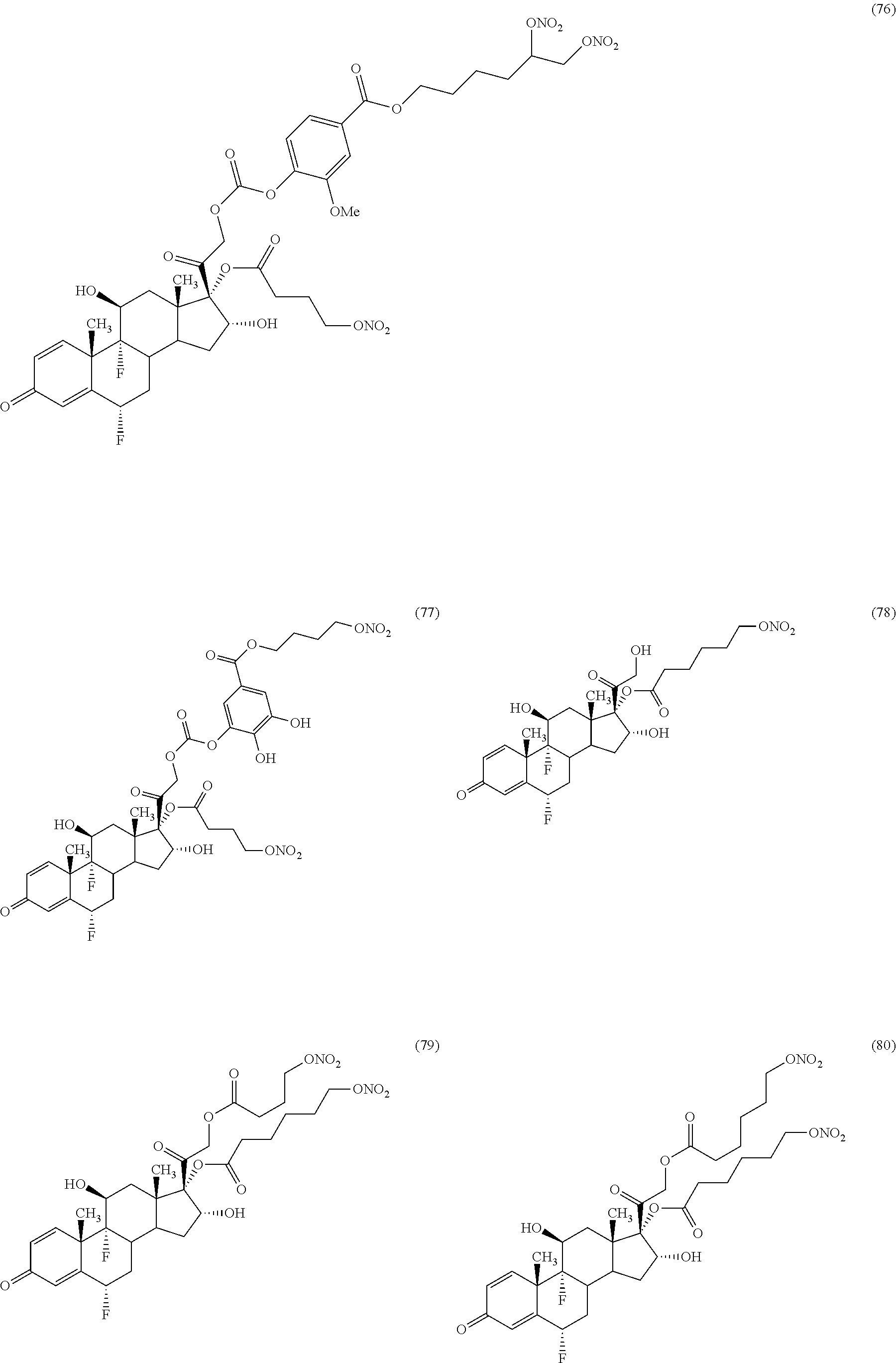

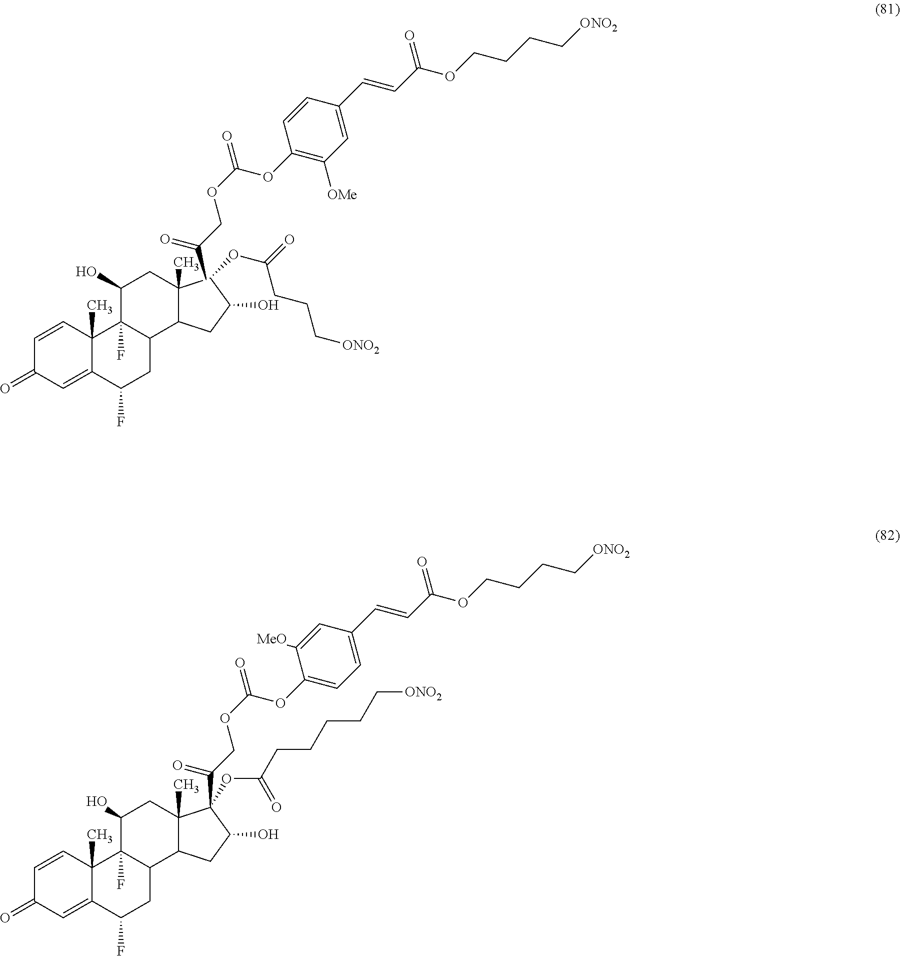

In another aspect of the invention, there is provided a compound of formula (I) for the treatment of inflammatory diseases.

In another aspect of the invention, there is provided a compound of formula (I) for the use in the prevention or in the treatment of ocular diseases, in particular diabetic macular edema, diabetic retinopathy, macular degeneration, age-related macular degeneration and other diseases of retina and macula lutea, in particular diabetic macular edema.

In yet another aspect of the invention, there is provided a pharmaceutical composition comprising a pharmaceutically effective amount of a compound of formula (I) and/or a salt or stereoisomer thereof and at lest an ophthalmically acceptable excipient in a suitable form for intravitreal or periorbital administration.

The term “excipient” is used herein to describe any ingredient other than the compound(s) of the invention. The choice of the excipient will to a large extent depend on factors such as the particular mode of administration, the effect of the excipient on the stability, and the nature of the dosage form.

In still another aspect of the invention, there is provided a pharmaceutical composition wherein the compound of the invention is administered as a suspension or emulsion in an ophthalmically acceptable vehicle.

The compounds of the invention intended for pharmaceutical use may be administered as crystalline or amorphous products. The compounds of the invention intended for pharmaceutical use may be administered alone or in combination with one or more other compounds of the invention.

The utility of the compounds of the invention as medical agents for the treatment or prevention of diabetic macula edema, diabetic retinopathy, macular degeneration, age-related macular degeneration and other diseases of retina and macula lutea is demonstrated by the activity of the compounds in conventional assays.

### Synthesis Procedure

In general the term “amino protecting group” as used herein refers to Boc, Fmoc or those described in T. W. Greene “Protective groups in organic synthesis”, Wiley-Interscience, 2007, 4^{th} edition,

the term “carboxylic protecting group” as used herein refers to tert-butyl ester and those described in T. W. Greene “Protective groups in organic synthesis”, Wiley-Interscience, 2007, 4^{th} edition,

The term “diol protecting group” as used herein refers to acetal, such as p-methoxybenzylidene, butylidene, and those described in T. W. Greene “Protective groups in organic synthesis”, Wiley-Interscience, 2007, 4^{nd} edition;

The term “hydroxyl protecting group” as used herein refers to silyl ethers, such as trimethylsilyl, tert-butyl-dimethylsilyl or trityl and those described in T. W. Greene “Protective groups in organic synthesis”, Wiley-Interscience, 2007, 4^{th} edition,

1) The compound of general formula (I) wherein R₄ is H, R₁, R₂, R₃, R₅ and R₆ are as above defined can be obtained:
1.1) by reacting a compound of formula (IIa), i.e. the precursor corticosteroid,

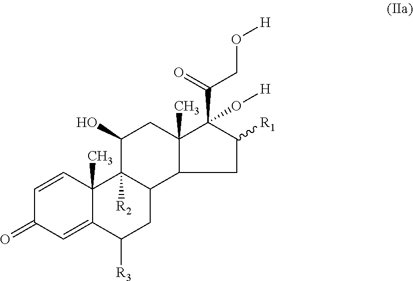

wherein R₁, R₂ and R₃ are as above defined,
with a compound of formula (IIIa)

(R_{A}O)₃C—R₅—CH(Q)R₆ (IIIa)

wherein:
R_{A} is straight alkyl C₁-C₁₀, R₅ and R₆ are as above defined and Q is ONO₂ or Q₁, wherein Q₁ is a chlorine atom, a bromine atom, a iodine atom, a mesyl group or a tosyl group; the reaction is carried out in the presence of an organic acid such as p-toluenesulfonic acid, in an inert organic solvent such as tetrahydrofuran, dioxane, at a temperature from −20° C. and 40° C. The reaction is completed within a time range from 30 minutes to 36 hours
and
1.2) hydrolyze the ortho ester of formula (IIb) obtained in 1.1)

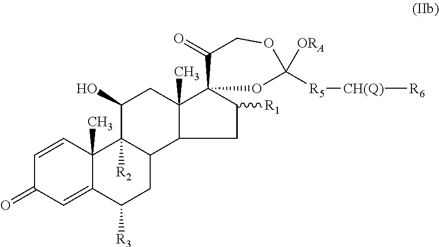

wherein R₁, R₂, R₃, R₅, R₆, R_{A} and Q are as above defined, by reacting the compound (IIb) with an organic acid such as AlCl₃, acetic acid, ossalic acid, in an organic aqueous solvent such as methanol, ethanol, propanol, isopropanol at a temperature from −20° C. and 40° C. The reaction is completed within a time range from 30 minutes to 36 hours
and
1.3) when Q is Q₁, by reacting the compound obtained in the step 1.2) with a nitrate source such as silver nitrate, lithium nitrate, sodium nitrate, potassium nitrate, magnesium nitrate, calcium nitrate, iron nitrate, zinc nitrate or tetraalkylammonium nitrate (wherein alkyl is C₁-C₁₀ alkyl) in a suitable organic solvent such as acetonitrile, tetrahydrofurane, methyl ethyl ketone, ethyl acetate, DMF; the reaction is carried out, in the dark, at a temperature from room temperature to the boiling temperature of the solvent. Alternatively, the reaction with AgNO₂ can be performed under microwave irradiation in solvents such acetonitrile or THF at temperatures in the range between about 100-180° C. for time range about 1-60 min. Preferred nitrate source is silver nitrate.

The compounds of formula (IIa) are commercially available

2) The compound of general formula (I) wherein R₁, R₂, R₃, R₅ and R₆ are as above defined, and
R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (F) —(Z)—Y

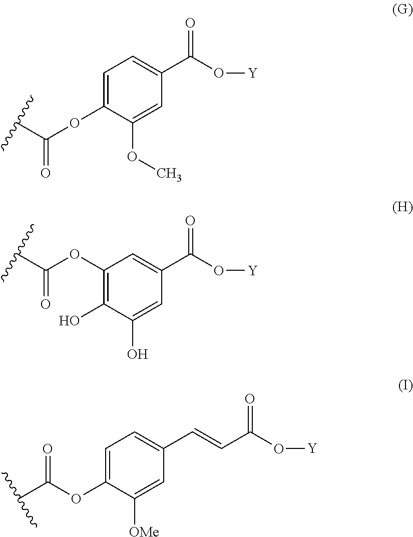

wherein
R¹ is selected from the group R^{1a}) as above defined,
R² is as above defined,

### Z is —C(O)O— and

Y is as above defined,
can be synthesized
2.1) by reacting a compound of formula (IIc)

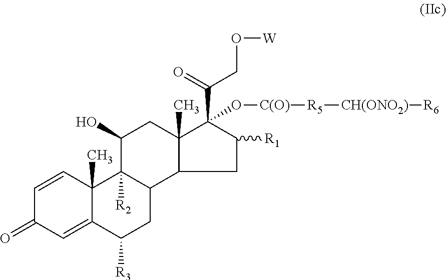

wherein R₁, R₂, R₃, R₅, R₆ are as above defined and W is H or COCl
with a compound of the following formulae:

### (A₁) W₁—R^{1a′}—CH(NHR^{2a})—C(O)—O—Y′

### (B₁) W₁—R^{1a′}—CH(COOP)NH—C(O)—Y′

### (C₁) W₁—R^{1a′}—CH(COOP)—O—C(O)—Y′

### (F₁) W₁—O—Y′

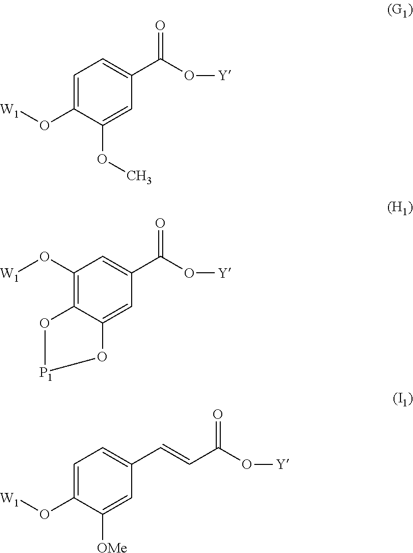

wherein
W₁ is —H or R_{B}OC(O)— wherein R_{B} is pentafluorophenyl, 4-nitrophenyl,
R^{1a′}) is selected from

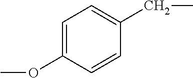

### —S—CH₂—, —O—CH(CH₃)—, —O—CH₂—;

R^{2a} is —H or —C(O)CH₃ or P₂ wherein P₂ is a amino protecting group,
P is a carboxylic protecting group, P₁ is a diol protective group,

### Y′ is:

### —R₇—CH(Q)R₈

—R₇—CH(Q)-(CR₉R₁₀)ₜ—CH(Q)R₈
—[(CH₂)ₒ—X]ₚ—[(CH₂)ᵣ—X]ₛ—(CH₂)_{q}—(CR₉R₁₀)ₜ—CH(Q)R₈
—[(CH₂)ₒ—X]ₚ—[(CH₂)ᵣ—X]ₛ—(CH₂)_{q}—CH(Q)-(CR₉R₁₀)ₜ—CH(Q)R₈
wherein
R₇, R₈, R₉, R₁₀, X, o, p, q, r, s and t are as above defined, Q is ONO₂ or Q₁ wherein Q₁ is selected from Cl, Br, I, a mesyl group or a tosyl group;
2.1.a) the reaction of a compound of formula (IIc) wherein W is H with a compound of formula (A₁), (B₁), (C₁), (F₁), (G₁), (H₁) or (I₁) wherein W₁ is R_{B}OC(O)— is carried out in presence of a catalyst, such as DMAP or in the presence of DMAP and a Lewis acid such as Sc(OTf)₃ or Bi(OTf)₃, in an inert organic solvent such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours;
2.1.b) The reaction of a compound of formula (IIc) wherein W is COCl with a compound of formula (A₁), (B₁), (C₁), (F₁), (G₁), (H₁) or (I₁) wherein W₁ is H may be carried out in presence of an organic base such as N,N-dimethylamino pyridine (DMAP), triethylamine, pyridine. The reaction is carried out in an inert organic solvent such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours;
2.2) when Q is Q₁, by reacting the compound obtained in the step 2.1) with a nitrate source according to the method described in 1.3)
and
2.3) optionally deprotecting the compounds obtained in step 2.1) or 2.2) as described in T. W. Greene “Protective groups in organic synthesis”, Wiley-Interscience, 2007, 4^{nd} edition. Trifluoroacetic acid or anhydrous inorganic acid are the preferred method for removing Boc protecting group, organic base such as piperidine is the preferred method for removing Fmoc protecting group. Aqueous or anhydrous organic or inorganic acid is the preferred method for removing t-butyl ester protecting group. Hydrochloric acid in tetrahydrofurane is the preferred method for removing acetal protecting group.

Alternatively the compound of general formula (I) as defined in 2), can be synthesized

3.1) by reacting a compound of formula (IIc) wherein R₁, R₂, R₃, R₅, R₆ and W are as above defined with a compound of formula

### (A₂) W₁—R^{1a′}—CH(NHR^{2a})—C(O)—O—P

### (B₂) W₁—R^{1a′}—CH(COOP)—NH—R^{2a}

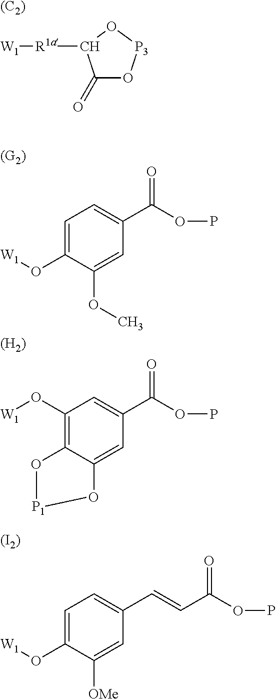

wherein
W₁ is —H or R_{B}OC(O)— wherein R_{B} is pentafluorophenyl, 4-nitrophenyl,
R^{1a′}, R^{2a}, R³, R⁴, P, P₁ are as above defined and P₃ is a alpha hydroxyl acid protecting group such as 4-oxo-1,3-dioxolane;
3.1.a) The reaction of a compound of formula (IIc) wherein W is H with a compound of formula (A₂), (B₂), (C₂), (G₂), (H₂), (I₂) wherein W₁ is R_{B}OC(O)— is carried out according to the method described in 2.1.a).
3.1.b) The reaction of a compound of formula (IIc) wherein W is COCl with a compound of formula (A₂), (B₂), (C₂), (G₂), (H₂), (I₂) wherein W₁ is H is carried out according to the method described in 2.1.b),
and
3.2) deprotecting the compounds obtained in steps 3.1.a)-3.1.b) as described in T. W. Greene “Protective groups in organic synthesis”, Wiley-Interscience, 2007, 4^{nd} edition, hydrochloric acid or anhydrous inorganic acid are the preferred method for removing alpha hydroxy acid protecting group,
and
3.3) by reacting a compound of formula (IId) obtained in the step 3.2)

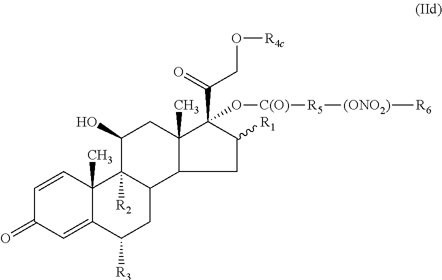

wherein R₁, R₂, R₃, R₅, R₆ are as above defined and R_{4c} is a radical selected from the following meaning

### (A₃) —R¹—CH(NHR^{2a})—C(O)OH

### (B₃) —R¹—CH(COOP)—NH₂

### (C₃) —R¹—CH(COOH)—OH

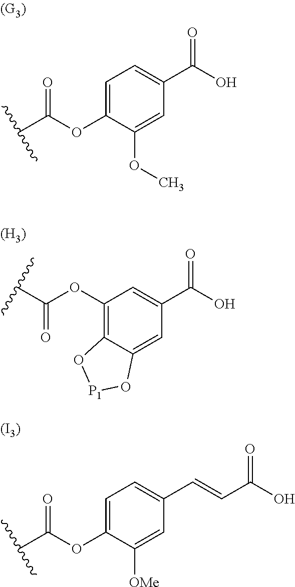

wherein
R¹ is selected from the group R^{1a}) as above defined, R^{2a} is as above defined,
with a compound of formula

### (VIa) W₂—R₇—CH(Q)R₈

(VIb) W₂—R₇—CH(Q)-(CR₉R₁₀)ₜ—CH(Q)R₈
(VIc) W₂—[(CH₂)ₒ—X]ₚ—[(CH₂)ᵣ—X]ₛ—(CH₂)_{q}—(CR₉R₁₀)ₜ—CH(Q)R₈
(VId) W₂—[(CH₂)ₒ—X]ₚ—[(CH₂)ᵣ—X]ₛ—(CH₂)_{q}—CH(Q)-(CR₉R₁₀)ₜ—CH(Q)R₈
wherein W₂ is selected from HO—, Cl, Br, I, —COOH, —COCl, —C(O)OR_{B} wherein R_{B} is as above defined;
W₂ is —OH, Cl, Br, I when R_{4c} is selected from (A₃), (G₃), (H₃), (I₃) or W₂ is —COOH, —C(O)OR_{B}, —CO—Cl when R_{4c} is selected from (B₃), (C₃);
3.3.a) the reaction of the compound of formula (IId) wherein R_{4c} is selected from (A₃), (G₃), (H₃), (I₃), with a compound of formula (VIa), (VIb), (VIc) or (VId) wherein W₂ is Cl, Br, I is carried out in the presence of a organic base such as 1,8-diazabiciclo[5.4.0]undec-7-ene (DBU), N,N-diisopropylethyl amine, diisopropylamine or an inorganic base such as alkaline-earth metal carbonate or hydroxide, potassium carbonate, cesium carbonate, in an inert organic solvent such as N,N′-dimethylformamide, tetrahydrofuran, acetone, methyl ethyl ketone, acetonitrile, a polyhalogenated aliphatic hydrocarbon at a temperature from −20° C. and 40° C., preferably from 5° C. to 25° C. The reaction is completed within a time range from 1 to 8 hours. When W₃ is chosen among chlorine or bromine the reaction is carried out in presence a iodine salts such as KI;
3.3.b) the reaction of a compound of formula (IId) wherein R_{4c} is a radical selected (A₃), (G₃), (H₃), (I₃), with a compound of formula (VIa), (VIb), (VIc) or (VId) wherein W₂ is —OH is carried out in the presence of a condensing agent such as dicyclohexylcarbodiimide (DCC), N′-(3-dimethyl aminopropyl)-N-ethylcarbodiimide hydrochloride (EDAC), N,N′-carbonyldiimidazole (CDI), optionally in the presence of a base, for example DMAP, in an inert organic solvent dry such as N,N′-dimethylformamide, tetrahydrofuran, benzene, toluene, dioxane, a polyhalogenated aliphatic hydrocarbon at a temperature from −20° C. and 50° C. The reaction is completed within a time range from 30 minutes to 36 hours;
3.3.c) the reaction of a compound of formula (IId) wherein R_{4c} is (B₃) or (C₃) with a compound of formula (VIa), (VIb), (VIc) or (VId) wherein W₂ is —COOH is carried out according to the method described in 3.3.b) or in presence of other condensing reagents such as O-(7-azabenzotriazol-1-yl)-N,N,N′,N′-tetramethyluronium hexafluorophosphate (HATU);
3.3.d) The reaction of a compound of formula (IId) wherein R_{4c} is (B₃) or (C₃) with a compound of formula (VIa), (VIb), (VIc) or (VId) wherein W₂ is —COCl may be carried out according to the method described in 2.1.b);
3.3.e) the reaction of a compound of formula (IId) wherein R_{4c} is (B₃) or (C₃) with a compound of formula (VIa), (VIb), (VIc) or (VId) wherein W₂ is R_{B}OC(O)— is carried out according to the method described in 2.1.a),
and
3.4) when Q is Q₁, by reacting the compound obtained in the steps 3.3.a)-3.3.e) according to the method described in 1.3)
and
3.5) deprotecting the compounds obtained in step 3.3) or 3.4) as described in T. W. Greene “Protective groups in organic synthesis”, Wiley-Interscience, 2007, 4^{nd} edition.
4) The compound of general formula (I) wherein R₁, R₂, R₃, R₅, R₆ are as above and R₄ is selected from:

### (A) —R¹—CH(NHR²)—C(O)—O—Y

### (B) —R¹—CH(COOH)NH—C(O)—Y

### (C) —R¹—CH(COOH)—O—C(O)—Y

### (D) —C(O)CH(R³)—NH—C(O)—Y

### (E) —C(O)CH₂—CH(R⁴)—NH—C(O)—Y

### (F) —(Z)—Y

wherein
R¹ is selected from the group R^{1b}) as above defined,
R², R³, R⁴ and Y are as above defined,

### Z is —C(O)—,

can be synthesized:
4.1) by reacting a compound of formula (IIc) as above defined wherein R₁, R₂, R₃, R₅, and R₆ are as above defined and W is H, with a compound a compound of formula:

### (A₄) W₃—R¹—CH(NHR^{2a})—C(O)—O—Y′

### (B₄) W₃—R¹—CH(COOP)NH—C(O)—Y′

### (C₄) W₃—R¹—CH(COOP)—O—C(O)—Y′

### (D₁) W₃—C(O)CH(R³)—NH—C(O)—Y′

### (E₁) W₃—C(O)CH₂—CH(R⁴)—NH—C(O)—Y′

### (F₂) W₃—(Z)—Y′

wherein W₃ is HO— or R_{B}O— wherein R_{B} is as above defined, R¹, R^{2a}, R³, R⁴, P and Y′ are as above defined;
4.1.a) the reaction of a compound of formula (IIc) wherein W is H with a compound of formula (A₄), (B₄), (C₄), (D₁), (E₁) or (F₂) wherein W₃ is R_{B}O— is carried out as reported in 2.1.a);
4.1.b) the reaction of a compound of formula (IIc) wherein W is H with a compound of formula (A₄), (B₄), (C₄), (D₁) (E₁) or (F₂) wherein W₃ is HO—, is carried out as reported in 3.3.b);
4.2) when Q is Q₁, by reacting the compound obtained in the step 4.1) with a nitrate source according to the method described in 1.3)
and
4.3) optionally deprotecting the compounds obtained in step 4.1) or 4.2) as described in T. W. Greene “Protective groups in organic synthesis”, Wiley-Interscience, 2007, 4^{nd} edition.

Alternatively the compound of general formula (I) as defined in 4) can be synthesized

4.4) by reacting a compound of formula (IIc) as above defined with a compound of formula

### (A₅) W₃—R¹—CH(NHR^{2a})—C(O)—O—P

### (B₅) W₃—R¹—CH(COOH)—NH—R^{2a}

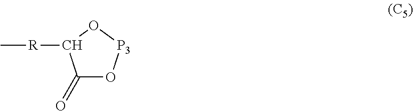

### (D₂) W₃—C(O)—CH(R³)—NH—R^{2a}

### (E₂) W₃—C(O)—CH₂—CH(R⁴)—NH—R^{2a}

wherein:
W₃, R¹, R^{2a}, R³, R⁴, P and P₃ are as above defined;
4.4.a) the reaction of a compound of formula (IIc) with a compound of formula (A₅), (B₅), (C₅), (D₂) or (E2) wherein W₃ is HO—, is carried out according to the method described in 4.1.b),
4.4.b) the reaction of a compound of formula (IIc) wherein W is H with a compound of formula (A₅), (B₅), (C₅), (D₂) or (E₂) wherein W₃ is R_{B}O— is carried out according to the method described in 4.1.a),
and
4.5) deprotecting the compounds obtained in step 4.4.a) or 4.4.b) as described in T. W. Greene “Protective groups in organic synthesis”, Wiley-Interscience, 2007, 4^{nd} edition,
and
4.6) by reacting a compound of formula (IIe) obtained in the step 4.5)

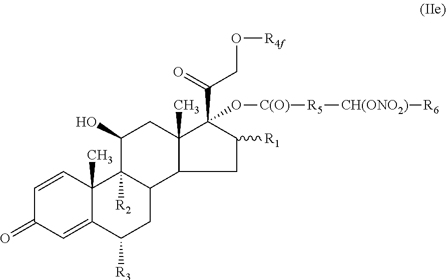

wherein R₁, R₂, R₃, R₅, R₆ are as above defined and R_{4f} is a radical selected from:

### (A₆) —R¹—CH(NHR^{2a})—C(O)OH

### (B₆) —R¹—CH(COOP)—NH₂

### (C₆) —R¹—CH(COOH)—OH

### (D₃) —C(O)—CH(R³)—NH₂

### (E₃) —C(O)—CH₂—CH(R⁴)—NH₂

wherein R¹ is selected from the group R^{1b}) as above defined, R^{2a}, R³, R⁴ and P are as above defined,
with a compound of formula

### (VIa) W₂—R₇—CH(Q)R₈

(VIb) W₂—R₇—CH(Q)-(CR₉R₁₀)ₜ—CH(Q)R₈
(VIc) W₂—[(CH₂)ₒ—X]ₚ—[(CH₂)ᵣ—X]ₛ—(CH₂)_{q}—(CR₉R₁₀)ₜ—CH(Q)R₈
(VId) W₂—[(CH₂)ₒ—X]ₚ—[(CH₂)ᵣ—X]ₛ—(CH₂)_{q}—CH(Q)-(CR₉R₁₀)ₜ—CH(Q)R₈
wherein
W₂ is HO—, Cl, Br, I when R_{4f} is (A₆), or W₂ is —COOH, —C(O)OR_{B} or —COCl when R_{4f} is (B₆), (C₆), (D₃) or (E₃);
4.6.a) the reaction of the compound of formula (IIe) wherein R_{4f} is (A₆), with a compound of formula (VIa), (VIb), (VIc), (VId) wherein W₂ is Cl, Br, I, is carried out according to the method described in 3.3.a);
4.6.b) the reaction of the compound of formula (IIe) wherein R_{4f} is (B₆), (C₆), (D₃) or (E₃) with a compound of formula (VIa), (VIb), (VIc), (VId) wherein W₂ is OH, is carried out according to the method described in 2.1.c).
4.6.c) the reaction of the compound of formula (IIe) wherein R_{4f} is (B₆), (C₆), (D₃) or (E₃) with a compound of formula (VIa), (VIb), (VIc), (VId) wherein W₂ is COOH is carried out according to the method described in 3.3.c);
4.6.d) The reaction of the compound of formula (IIe) wherein R_{4f} is (B₆), (C₆), (D₃) or (E₃) with a compound of formula (VIa), (VIb), (VIc), (VId) wherein W₂ is COCl may be carried out according to the method described in 2.1.b);
4.6.e) the reaction of the compound of formula (IIe) wherein R_{4f} is (B₆), (C₆), (D₃) or (E₃) with a compound of formula (VIa), (VIb), (VIc), (VId) wherein W₂ is —C(O)OR_{B} is carried out according to the method described in 2.1.a),
and
4.7) when Q is Q₁, by reacting the compound obtained in steps 4.6.a)-4.6.e) according to the method described in 1.3)
and
4.8) deprotecting the compounds obtained in step 4.6) or 4.7) as described in T. W. Greene “Protective groups in organic synthesis”, Wiley-Interscience, 2007, 4^{nd} edition.
5) Preparation of compound (IIc)

The compounds of formula (IIc) wherein R₁, R₂, R₃, R₅ and R₆ are as above defined and W is —COCl are prepared starting from the compounds obtained in 1.3), according to methods known in the literature.

6) Preparation of compound (IIIa)

The compounds of formula (IIIa) wherein R_{A}, R₅, R₆ are as above defined and Q is Q₁ are commercially available or can be obtained according to methods known in the literature. The compounds of formula (IIIa) wherein R_{A}, R₅, R₆ are as above defined and Q is ONO₂ can be obtained by reacting the compound (IIIa) wherein Q is Q₁ with a nitrate source as above described.

7) Preparation of the following compounds

### (A₁) W₁—R^{1a′}—CH(NHR^{2a})—C(O)—O—Y′

### (B₁) W₁—R^{1a′}—CH(COOP)NH—C(O)—Y′

### (C₁) W₁—R^{1a′}—CH(COOP)—O—C(O)—Y′

### (A₄) W₃—R¹—CH(NHR^{2a})—C(O)—O—Y′

### (B₄) W₃—R^{1a′}—CH(COOP)NH—C(O)—Y′

### (C₄) W₃—R¹—CH(COOP)—O—C(O)—Y′

### (D₁) W₃—C(O)CH(R³)—NH—C(O)—Y′

### (E₁) W₃—C(O)CH₂—CH(R⁴)—NH—C(O)—Y′

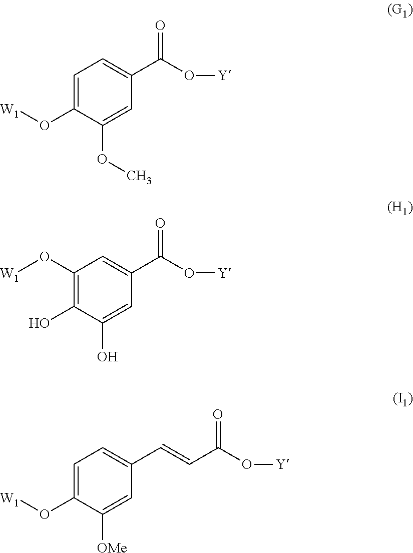

wherein

### W₁ is H, W₃ is —OH,

R^{1a′}, R^{2a}, R³, R⁴, P and Y′ are as above defined and
R¹ is selected from the group R^{1b}) as above defined,
can be obtained synthesized
7.1) by reacting a compound of formula

### (A₇) P₄—R^{1a′}—CH(NHR^{2a})—C(O)—OH

### (A₈) PO—R¹—CH(NHR^{2a})—C(O)—OH

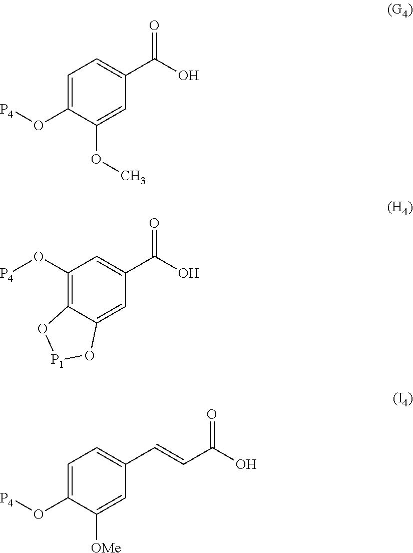

wherein
P, P₁, R^{1a′}, R^{2a} are as above defined,
R¹ is selected from the group R^{1b}) as above defined,
P₄ is a hydroxyl protecting group,
with a compound of formula

### (VIa) W₂—R₇—CH(Q)R₈

(VIb) W₂—R₇—CH(Q)-(CR₉R₁₀)ₜ—CH(Q)R₈
(VIc) W₂—[(CH₂)ₒ—X]ₚ—[(CH₂)ᵣ—X]ₛ—(CH₂)_{q}—(CR₉R₁₀)ₜ—CH(Q)R₈
(VId) W₂—[(CH₂)ₒ—X]ₚ—[(CH₂)ᵣ—X]ₛ—(CH₂)_{q}—CH(Q)-(CR₉R₁₀)ₜ—CH(Q)R₈
wherein
Q, X, o, p, r, s, t, R₇, R₈, R₉, R₁₀ are as above defined, W₂ is HO—, Cl, Br, I,
7.1.a) the reaction of a compound of formula (A₇), (A₈) (G₄), (H₄), (I₄) with a compound of formula (VIa) (VIb), (VIc), (VId) wherein W₂ is Cl, Br, I is carried out according to the method described in 3.3.a)
7.1.b) The reaction of a compound of formula (A₇), (A₈) (G₄), (H₄), (I₄) with a compound of formula (VIa) (VIc), (VId) wherein W₂ is OH is carried out according to the method described in 2.1.c).
7.2) or by reaction a compound of formula

### (B₇) P₄—R^{1a′}—CH(COOP)—NH₂

### (C₇) P₄—R^{1a′}—CH(COOH)—OH

### (D₄) POC(O)—CH(R³)—NH₂

### (E₄) POC(O)—CH₂—CH(R⁴)—NH₂

### (B₈) PO—R¹—CH(COOP)—NH₂

### (C₈) PO—R¹—CH(COOH)—OH

wherein
P, R^{1a′}, R³, R⁴ and P are as above defined and
P₄ is a hydroxyl protecting group,
R¹ is selected from the group R^{1b} as above defined,
with a compound of formula

### (VIa) W₂—R₇—CH(Q)R₈

(VIb) W₂—R₇—CH(Q)-(CR₉R₁₀)ₜ—CH(Q)R₈
(VIc) W₂—[(CH₂)ₒ—X]ₚ—[(CH₂)ᵣ—X]ₛ—(CH₂)_{q}—(CR₉R₁₀)ₜ—CH(Q)R₈
(VId) W₂—[(CH₂)ₒ—X]ₚ—[(CH₂)ᵣ—X]ₛ—(CH₂)_{q}—CH(Q)-(CR₉R₁₀)ₜ—CH(Q)R₈
wherein
Q, X, o, p, r, s, t, R₇, R₈, R₉, R₁₀ are as above defined, W₂ is —COOH, —COCl or R_{B}OC(O)— wherein R_{B} is as above defined;
7.2.a) the reaction of a compound of formula (B₇), (B₈), (C₇), (C₈), (D₄), (E₄) with a compound of formula (VIa), (VIb), (VIc), (VId) wherein W₂ is COOH is carried out according to the method described in 3.3.c),
7.2.b) the reaction of a compound of formula B₇), (B₈), (C₇), (C₈), (D₄), (E₄) with a compound of formula (VIa) (VIb), (VIc), (VId) wherein W₂ is —COCl is carried out according to the method described in 2.1.b).
7.2.c) the reaction of a compound of formula B₇), (B₈), (C₇), (C₉), (D₄), (E₄)) with a compound of formula (VIa), (VIb), (VIc), (VId) wherein W₂ is R_{B}OC(O)— is carried out according to the method described in 2.1.a),
and
7.3) when Q is Q₁, by reacting the compound obtained in the steps 6.1.a), 6.1.b), 6.2.a)-6.2.c) with a nitrate source according to the method described in 1.3)
and
7.4) deprotecting the compounds obtained in steps 6.1) and 6.2) or 6.3) as described in T. W. Greene “Protective groups in organic synthesis”, Wiley-Interscience, 2007, 4^{nd} edition. Fluoride ion is the preferred method for removing the silyl ether group.

The compounds of formula (A₇), (A₈), (B₇), (B₈), (C₇), (C₈), (D₄), (E₄), (G₄), (H₄), (I₄) are commercially available or can be obtained according to methods known in the literature

8) The compounds of formula

### (A₄) W₃—R¹—CH(NHR^{2a})—C(O)—O—Y′

### (B₄) W₃—R¹—CH(COOP)NH—C(O)—Y′

### (C₄) W₃—R¹—CH(COOP)—O—C(O)—Y′

### (D₁) W₃—C(O)CH(R³)—NH—C(O)—Y′

### (E₁) W₃—C(O)CH₂—CH(R⁴)—NH—C(O)—Y′

wherein W₃ is R_{B}O—, R¹ is selected from the group R^{1b}), R^{2a}, R³, R⁴ P and Y′ are as above defined can be synthesized according to methods known in the literature from the correspondent compounds of formula (A₄), (B₄), (C₄), (D₁), (E₁) wherein W₃ is —OH.
9) The compounds of formula (VIa), (VIb), (VIc), (VId) are commercially available or can be obtained according to methods as known in the literature.

### EXAMPLE 1

### Compound (1)

### Synthesis of (9R,10S,11S,13S,16S,17R)-9-fluoro-11-hydroxy-17-(2-hydroxyacetyl)-10,13,16-trimethyl-3-oxo-6,7,8,9,10,11,12,13,14,15,16,17-dodecahydro-3H-cyclopenta[a]phenanthren-17-yl 4-(nitrooxy)butanoate

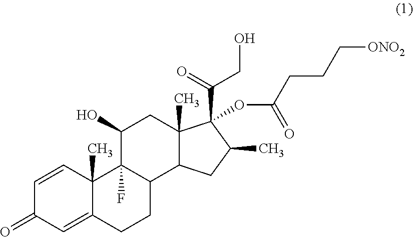

### A) (4′R,9R,10S,11S,13S,16S)-2′-(3-bromopropyl)-9-fluoro-11-hydroxy-2′-methoxy-10,13,16-trimethyl-7,8,9,10,11,12,13,14,15,16-decahydrospiro[cyclopenta[a]phenanthrene-17,4′-[1,3]dioxane]-3,5′(6H)-dione

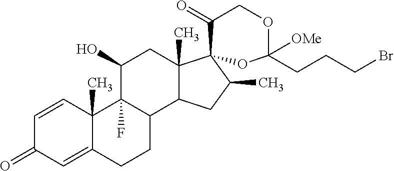

To a solution of betamethasone (1.0 g, 2.54 mmol) in toluene (14 ml) and N,N-dimethylformamide (2 ml), p-toluenesulfonic acid (cat) and trimethyl-4-bromo-orthobutyrate (0.88 ml, 5.09 mmol) were added. The reaction was stirred at room temperature for 25 hours. The mixture was poured in water (30 ml) and extracted with ethyl acetate (40×4 ml), the organic layers were dried over sodium sulfate and concentrated under reduced pressure. The residue was purified by flash chromatography, (Biotage System, column FLASH 40+M™ KP-Sil, eluent: gradient n-hexane/ethyl acetate 9/1 (200 ml), to n-hexane/ethyl acetate 3/7 during 1400 ml, n-hexane/ethyl acetate 3/7 (200 ml)). The product (1.08 g) was obtained.

### B) (9R,10S,11S,13S,16S,17R)-9-fluoro-11-hydroxy-17-(2-hydroxyacetyl)-10,13,16-trimethyl-3-oxo-6,7,8,9,10,11,12,13,14,15,16,17-dodecahydro-3H-cyclopenta[a]phenanthren-17-yl 4-(bromo)butanoate

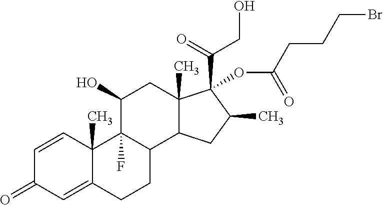

To a solution of compound A (1.08 g, 1.94 mmol) in methanol (35 ml), a 5% aqueous AcOH solution (6.9 ml) was added. The reaction was stirred a reflux for 4 hours. The mixture was concentrated under reduced pressure. The mixture was diluted with dichloromethane (25 ml), washed with saturated aqueous sodium carbonate (2×30 ml), water (2×30 ml), the organic layers were dried over sodium sulfate and concentrated under reduced pressure. The product (0.95 g) was obtained.

### C) (9R,10S,11S,13S,16S,17R)-9-fluoro-11-hydroxy-17-(2-hydroxyacetyl)-10,13,16-trimethyl-3-oxo-6,7,8,9,10,11,12,13,14,15,16,17-dodecahydro-3H-cyclopenta[a]phenanthren-17-yl 4-(nitrooxy)butanoate

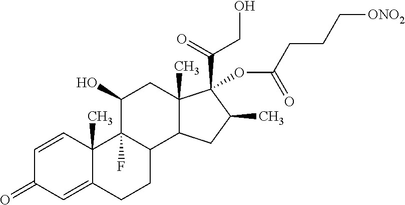

To a solution of compound B (0.42 g, 0.78 mmol) in acetonitrile (18 ml), silver nitrate (0.39 g, 2.35 mmol) was added. The reaction was heated to 120° C. for 10 minutes under microwave irradiation. The resulting mixture was cooled, filtered and the solvent was removed under reduced pressure. The residue was purified by flash chromatography (Biotage System, SNAP Cartridge silica 100 g, eluent: gradient n-hexane/ethyl acetate 9/1 (200 ml), to n-hexane/ethyl acetate 3/7 during 1200 ml, n-hexane/ethyl acetate 3/7 (600 ml)). The product (0.56 g) was obtained.

¹H-NMR: (DMSO), δ: 7.28 (1H, d); 6.23 (1H, dd); 6.02 (1H, s); 5.43 (1H, s); 4.96 (1H, s); 4.52 (2H, t), 4.20 (1H, m); 3.91 (2H, m); 2.80-2.30 (4H, m); 2.30-2.07 (2H, m); 1.89-1.75 (4H, m); 1.59-1.49 (1H, m); 1.49 (3H, s); 1.37-1.06 (4H, m); 1.27 (3H, d); 0.85 (3H, s).

### EXAMPLE 2

### Compound (2)

### Synthesis of (9R,10S,11S,13S,16S,17R)-9-fluoro-11-hydroxy-10,13,16-trimethyl-17-(2-(4-(nitrooxy)butanoyloxy)acetyl)-3-oxo-6,7,8,9,10,11,12,13,14,15,16,17-dodecahydro-3H-cyclopenta[a]phenanthren-17-yl 4-(nitrooxy)butanoate

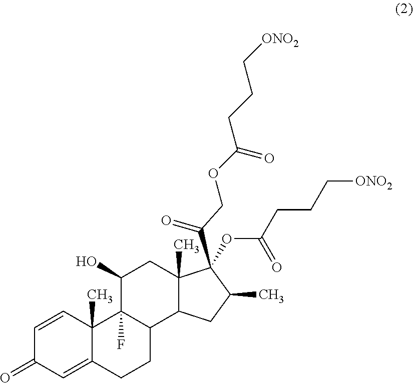

To a solution of compound C (1.0 g, 1.91 mmol) in dichloromethane (50 ml), DMAP (0.34 g, 2.86 mmol) was added. The reaction was cooled at 0° C. and 4-nitrooxybutanoic acid pentafluorophenol ester (0.60 g, 1.91 mmol) was added. The reaction was stirred at room temperature for 16 hours. The solvent was evaporated under vacuum. The residue was purified by flash chromatography (Biotage System, SNAP Cartridge silica 100 g, eluent: gradient n-hexane/ethyl acetate 9/1 (200 ml), to n-hexane/ethyl acetate 3/7 during 1200 ml, n-hexane/ethyl acetate 3/7 (600 ml)). The product (1.16 g) was obtained.

¹H-NMR: (DMSO), δ: 7.29 (1H, d); 6.22 (1H, dd); 6.03 (1H, s); 5.55 (1H, d); 4.80-4.49 (6H, m); 4.22 (1H, bs); 2.70-1.65 (17H, m); 1.49 (3H, s); 1.50-1.30 (1H, m); 1.22 (3H, d); 1.29-1.05 (1H, m); 0.86 (3H, s).

### EXAMPLE 3

### Compound (3)

### Synthesis of 4-(nitrooxy)butyl 4-((2-((9R,10S,11S,13S,16S,17R)-9-fluoro-11-hydroxy-10,13,16-trimethyl-17-(4-(nitrooxy)butanoyloxy)-3-oxo-6,7,8,9,10,11,12,13,14,15,16,17-dodecahydro-3H-cyclopenta[a]phenanthren-17-yl)-2-oxoethoxy)carbonyloxy)-3-methoxybenzoate

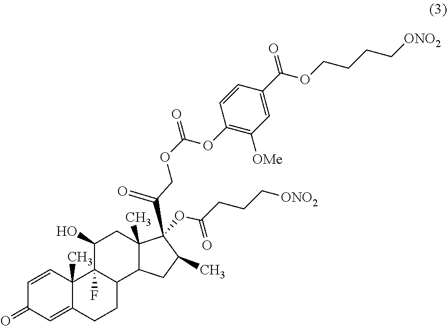

### D) (9R,10S,11S,13S,16S,17R)-17-(2-(chlorocarbonyloxy)acetyl)-9-fluoro-11-hydroxy-10,13,16-trimethyl-3-oxo-6,7,8,9,10,11,12,13,14,15,16,17-dodecahydro-3H-cyclopenta[a]phenanthren-17-yl 4-hydroxybutanoate

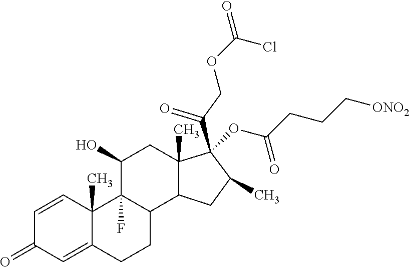

To a solution of compound C (1.0 g, 1.91 mmol) in tetrahydrofurane (12 ml), cooled at 0° C. and under N₂, a 20% toluene solution of phosgene (6.08 ml, 11.46 mmol) was added. The reaction was stirred at 0° C. for 1 hour and at room temperature for 21 hours. The excess of phosgene was removed by heating at 40° C. for 45 minutes. The solvent was evaporated under vacuum. The mixture was diluted with dichloromethane (50 ml), washed with water (3×25 ml). The organic layer was dried over sodium sulfate and concentrated under reduced pressure. The product (1.09 g) was obtained.

### E) 4-(nitrooxy)butyl 4-((2-((9R,10S,11S,13S,16S,17R)-9-fluoro-11-hydroxy-10,13,16-trimethyl-17-(4-(nitrooxy)butanoyloxy)-3-oxo-6,7,8,9,10,11,12,13,14,15,16,17-dodecahydro-3H-cyclopenta[a]phenanthren-17-yl)-2-oxoethoxy)carbonyloxy)-3-methoxybenzoate

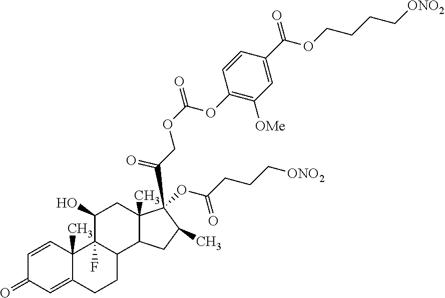

To a solution of compound D (1.09 g, 1.86 mmol) in dichloromethane (19 ml), diisopropylethylamine (0.35 ml, 2.04 mmol) was added. The reaction was cooled at 0° C. and vanillic acid 4-(nitrooxy)butyl ester (0.58 g, 2.04 mmol) was added. The reaction was stirred at room temperature for hours. The solvent was evaporated under vacuum. The residue was purified by flash chromatography (Biotage System, SNAP Cartridge silica 100 g, eluent: n-hexane/ethyl acetate 85/15 (200 ml)). The product (0.91 g) was obtained.

¹H-NMR: (DMSO), δ: 7.63 (2H, dd); 7.40 (1H, d); 7.26 (1H, d); 6.23 (1H, dd); 6.02 (1H, s); 5.49 (1H, d); 4.78 (2H, m); 4.60 (2H, t); 4.53 (2H, t); 4.31 (2H, m); 4.20 (1H, bs); 3.88 (3H, s); 3.48 (3H, s); 2.80-2.30 (4H, m); 2.30-2.07 (2H, m); 1.99 (3H, m); 1.89-1.75 (4H, m); 1.59-1.49 (1H, m); 1.37-1.06 (4H, m); 1.13 (3H, d); 0.87 (3H, s).

### EXAMPLE 4

### Compound (4)

### Synthesis of (9R,10S,11S,13S,16S,17R)-17-(2-((4-((S)-2-acetamido-3-(4-(nitrooxy)butoxy)-3-oxopropyl)phenoxy)carbonyloxy)acetyl)-9-fluoro-11-hydroxy-10,13,16-trimethyl-3-oxo-6,7,8,9,10,11,12,13,14,15,16,17-dodecahydro-3H-cyclopenta[a]phenanthren-17-yl 4-(nitrooxy) butanoate

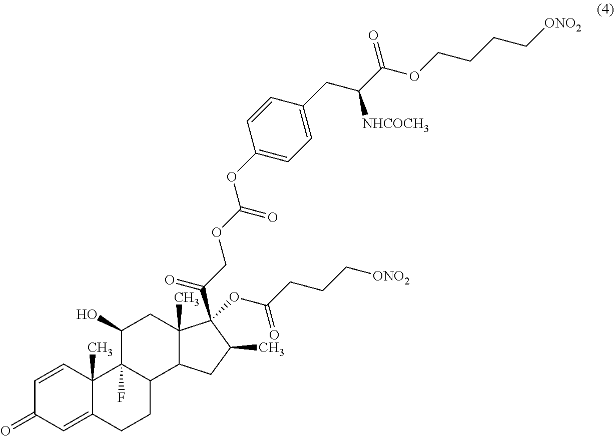

### F) (S)-4-(nitrooxy)butyl 2-(tert-butoxycarbonylamino)-3-(4-hydroxyphenyl)propanoate

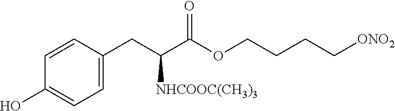

To a solution of Boc-L-tyrosine (4.2 g, 15.07 mmol) in N,N-dimethylformamide (34 ml), cesium carbonate (4.92 g, 15.07 mmol) was added. The reaction was cooled at 0° C. and a 20% solution of 1-bromo-4-(nitrooxy)butane in dichloromethane (14.96 g) was added. The reaction was stirred at room temperature for 22 hours. The mixture was poured into a 5% aqueous NaH₂PO₄ solution and extracted with diethyl ether (3×50 ml). The organic layers were washed with water (50 ml), dried over sodium sulfate and concentrated under reduced pressure. The residue was purified by flash chromatography (Biotage System, Cartridge column FLASH 40+M™ KP-Sil, eluent: gradient n-hexane/ethyl acetate 9/1 (200 ml), to n-hexane/ethyl acetate 3/7 during 1200 ml, n-hexane/ethyl acetate 7/3 (200 ml)). The product (5.32 g) was obtained.

### G) (S)-4-(nitrooxy)butyl 2-amino-3-(4-hydroxyphenyl) propanoate

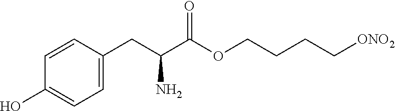

HCl gas was bubbled through a solution of compound F (3.16 g, 7.89 mmol) in dichloromethane (55 ml) for 20 minutes. The mixture was poured into a saturated aqueous NaHCO₃ solution (50 ml). The organic layer separated was dried over sodium sulfate and concentrated under reduced pressure. The crude product (2.76 g) was used in the next step without any purification.

### H) (S)-4-(nitrooxy)butyl 2-acetylamino-3-(4-hydroxyphenyl) propanoate

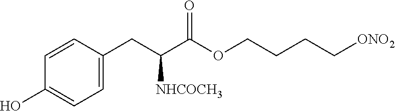

To a solution of compound G) (2.35 g, 7.89 mmol) in dichloromethane (35 ml), cooled at 0° C., N,N-diisopropylethylamine (1.39 ml, 7.89 mmol) and acetyl chloride (0.617 ml, 8.68 mmol) were added. The reaction was stirred at room temperature for 17 hours. The mixture was washed with water (3×50 ml); the organic layer was dried over sodium sulfate and concentrated under reduced pressure. The residue was purified by flash chromatography (Biotage System, SNAP Cartridge silica 100 g, eluent: gradient dichloromethane/acetone 9/1 (200 ml), to dichloromethane/acetone 8/2 during 1400 ml, dichloromethane/acetone 8/2 (500 ml)). The product (0.786 g) was obtained.

### I) (9R,10S,11S,13S,16S,17R)-17-(2-((4-((S)-2-acetamido-3-(4-(nitrooxy)butoxy)-3-oxopropyl)phenoxy)carbonyloxy)acetyl)-9-fluoro-11-hydroxy-10,13,16-trimethyl-3-oxo-6,7,8,9,10,11,12,13,14,15,16,17-dodecahydro-3H-cyclopenta[a]phenanthren-17-yl 4-(nitrooxy)butanoate

To a solution of compound D (0.569 g, 0.97 mmol) in dichloromethane (15 ml), diisopropylethylamine (0.18 ml, 1.06 mmol) was added. The reaction was cooled at 0° C. and a solution of compound H (0.36 g, 1.06 mmol) in dichloromethane (3 ml) was added. The reaction was stirred at room temperature for 18 hours. The solvent was evaporated under vacuum. The residue was purified by flash chromatography (Biotage System, SNAP Cartridge silica 100 g, eluent: gradient dichloromethane/acetone 9/1 (200 ml), to dichloromethane/acetone 8/2 during 1000 ml, dichloromethane/acetone 8/2 (1000 ml)). The product (0.477 g) was obtained.

¹H-NMR: (DMSO), δ: 8.36 (1H, d); 7.29 (3H, m); 7.15 (2H, d); 6.23 (1H, dd); 6.02 (1H, s); 5.52 (1H, d); 4.75 (2H, m); 4.49 (4H, m); 4.39 (1H, m); 4.20 (1H, bs); 4.04 (2H, t); 3.03-2.88 (2H, m); 2.70-1.00 (19H, m); 1.81 (3H, s); 1.48 (3H, s); 1.32 (3H, d); 0.88 (3H, s).

### EXAMPLE 5

### Compound (5)

### Synthesis of (9R,10S,11S,13S,16S,17R)-17-(2-((4-((S)-2-amino-3-(4-(nitrooxy)butoxy)-3-oxopropyl)phenoxy) carbonyloxy)acetyl)-9-fluoro-11-hydroxy-10,13,16-trimethyl-3-oxo-6,7,8,9,10,11,12,13,14,15,16,17-dodecahydro-3H-cyclopenta[a]phenanthren-17-yl 4-(nitrooxy)butanoate

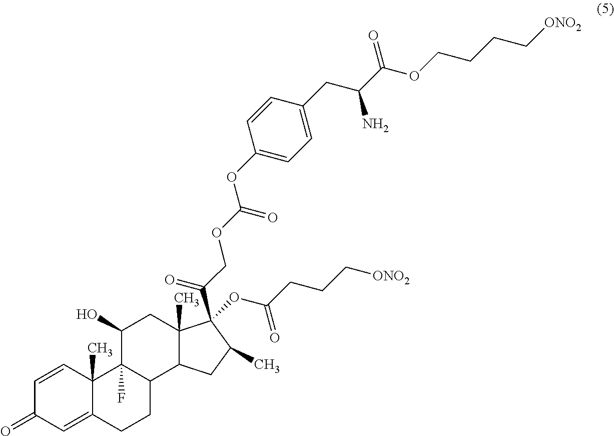

### L) (9R,10S,11S,13S,16S,17R)-17-(2-((4-((S)-2-(tert-butoxy carbonylamino)-3-(4-(nitrooxy)butoxy)-3-oxopropyl)phenoxy)carbonyloxy)acetyl)-9-fluoro-11-hydroxy-10,13,16-trimethyl-3-oxo-6,7,8,9,10,11,12,13,14,15,16,17-dodecahydro-3H-cyclopenta[a]phenanthren-17-yl-4-(nitrooxy)butanoate

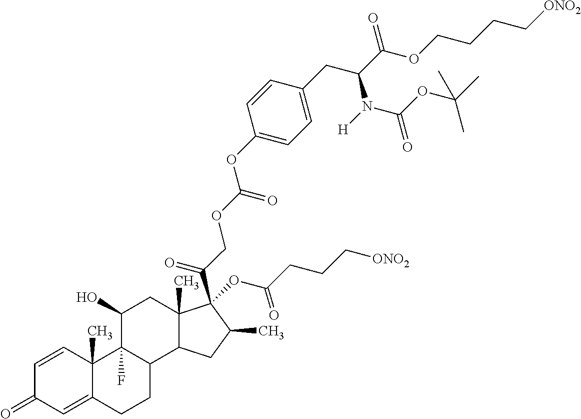

To a solution of compound D (0.569 g, 0.97 mmol) in dichloromethane (15 ml), diisopropylethylamine (0.18 ml, 1.06 mmol) was added. The reaction was cooled at 0° C. and a solution of compound F (0.42 g, 1.06 mmol) in dichloromethane (3 ml) was added. The reaction was stirred at room temperature for 19 hours. The solvent was evaporated under vacuum. The residue was purified by flash chromatography (Biotage System, SNAP Cartridge silica 100 g, eluent: gradient n-hexane/ethyl acetate 9/1 (200 ml), to n-hexane/ethyl acetate 3/7 during 1000 ml, n-hexane/ethyl acetate 3/7 (500 ml)). The product (0.7 g) was obtained.

### M) (9R,10S,11S,13S,16S,17R)-17-(2-((4-((S)-2-amino-3-(4-(nitrooxy)butoxy)-3-oxopropyl)phenoxy)carbonyloxy)acetyl)-9-fluoro-11-hydroxy-10,13,16-trimethyl-3-oxo-6,7,8,9,10,11,12,13,14,15,16,17-dodecahydro-3H-cyclopenta[a]phenanthren-17-yl 4-(nitrooxy)butanoate

HCl gas was bubbled through a solution of compound F (0.7 g, 0.73 mmol) in dichloromethane (15 ml) for 20 minutes. The mixture was poured into a saturated aqueous NaHCO₃ solution (30 ml). The organic layer separated was dried over sodium sulfate and concentrated under reduced pressure. The residue was purified by flash chromatography (Biotage System, Cartridge column FLASH 25+M™ KP-Sil, eluent: gradient dichloromethane/acetone 9/1 (200 ml), to dichloromethane/acetone 1/1 during 1400 ml). The product as free base (0.554 g) was obtained.

¹H-NMR: (DMSO), δ: 7.27 (3H, m); 7.13 (2H, d); 6.23 (1H, dd); 6.02 (1H, s); 5.51 (1H, d); 4.74 (2H, m); 4.49 (4H, m); 4.32 (1H, m); 4.20 (1H, bs); 4.02 (2H, t); 2.83-2.75 (2H, m); 2.70-1.00 (19H, m); 1.48 (3H, s); 1.32 (3H, d); 0.88 (3H, s).

### Assay on Vascular Tone

### Test Compounds:

Compound (2) described in Ex. 2
Compound (3) described in Ex. 3

### Reference Compounds:

### Betamethasone

The ability of the compounds of the invention to induce vasorelaxation in comparison to precursor compound, was tested in vitro in isolated rabbit thoracic aorta preparations (Wanstall J. C. et al., Br. J. Pharmacol., 134:463-472, 2001).

Male New Zealand rabbits (1, 8-2 Kg) were used. The animals were anaesthetized with sodium thiopental (50 mg/kg, iv), sacrificed by exsanguinations and then the thorax was opened and the aorta dissected. The aortas were placed immediately in Krebs-HEPES buffer (pH 7.4; composition mM: NaCl 130.0, KCl 3.7, NaHCO₃ 14.9, KH₂PO₄ 1.2, MgSO₄⊙7H₂O 1.2, Glucose 11.0, HEPES 10.0, CaCl₂.2H₂O 1.6) and cut into ring segments (4-5 mm in length). Each ring was placed in a 5 ml tissue bath filled with Krebs-HEPES buffer (37° C.) aerated with 95% O₂ and 5% CO₂ and was then attached to a force transducer (Grass FT03), connected to a BIOPAC MP150 System for measurement of the isometric tension². The preparations were allowed to equilibrate for 1 h at a resting tension of 2 g with changes of the buffer every 15 minutes and then stimulated by exposure to 90 mM KCl (3 times) with intervening washings. After equilibration, the rings were precontracted submaximally with methoxamine (3 μM) and, when the contraction reach a steady state a cumulative concentration-response curve to the test compounds was obtained. The time intervals between doses were based on the time needed to reach a full a steady state response.

Responses to test compounds were expressed as a percentage of residual contraction and plotted against concentration of test compound. EC₅₀ values (where EC₅₀ is the concentration producing 50% of the maximum relaxation to the test compound) were interpolated from these plots.

As shown in Table 1, the test compounds were able to induce relaxation in a concentration-dependent manner.

TABLE 1
Assay on vascular tone

| Test Compound | EC₅₀ (μM) |
|---|---|
| betamethasone | no effect |
| Compound (2) | 1.68 ± 0.61 |
| Compound (3) | 1.14 ± 0.48 |

1. A compound of formula (I) and pharmaceutically acceptable salts or stereoisomers thereof 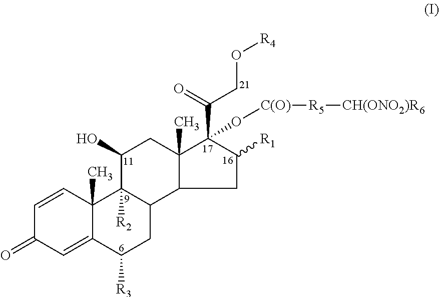
wherein
R₁ is CH₃ or OH, R₂ is F or Cl and R₃ is H or F, with the proviso that:
when R₁ is CH₃ then R₃ is H,
when R₁ is OH then R₂ is F;
when R₁ is CH₃, the CH₃ is linked to the carbon atom 16 in β position,
when R₁ is OH, the OH is linked to the carbon atom 16 in α position;
R₅ is a straight or branched C₁-C₁₀ alkylene;
R₆ is H or a straight or branched C₁-C₄ alkyl;
R₄ is —H or R₄ is selected from:
(A) —R¹—CH(NHR²)—C(O)—O—Y
(B) —R¹—CH(COOH)NH—C(O)—Y
(C) —R¹—CH(COOH)—O—C(O)—Y
(D) —C(O)CH(R³)—NH—C(O)—Y
(E) —C(O)CH₂—CH(R⁴)—NH—C(O)—Y
(F) —(Z)—Y 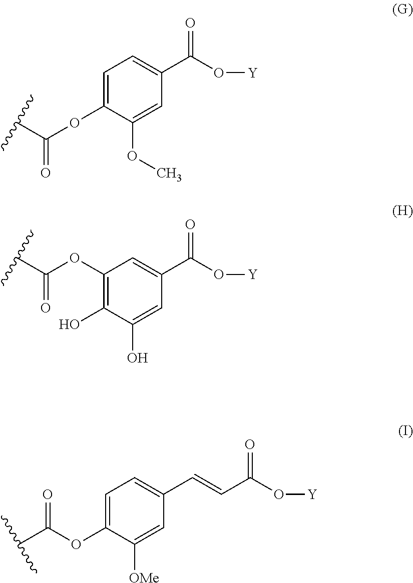
wherein:
R¹ is selected from:
R^{1a}) 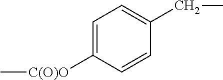
—C(O)—S—CH₂—, —C(O)O—CH(CH₃)—, —C(O)O—CH₂—;
R^{1b})
—C(O)—CH₂—, —C(O)—(CH₂)₂—;
R² is —H or —C(O)CH₃;
R³ is —H, —CH₃, isopropyl, isobutyl, sec-butyl,
methylthio-(CH₂)₂—, benzyl;
R⁴ is —H, —CH₃, isopropyl, isobutyl, sec-butyl, methylthio-(CH₂)₂—, benzyl;
Z is —C(O) or —C(O)—X″, wherein X″ is O, S or NR₁₂ wherein R₁₂ is H or a C₁-C₄ alkyl;
Y is selected from
—R₇—CH(ONO₂)R₈
—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene;
R₈ is H or a straight or branched C₁-C₄ alkyl;
R₉ and R₁₀ at each occurrence are independently H or a straight or branched C₁-C₁₀ alkylene;
o and r are integers from 1 to 6;
p and s are integers from 1 to 6;
q is an integer from 0 to 6;
t is an integer from 0 to 6;
X is O, S or NR₁₁ wherein R₁₁ is H or a C₁-C₄ alkyl.

2. A compound according to claim 1 wherein R₄ is —H.

3. A compound according to claim 2 wherein
R₁ is CH₃ linked to the carbon atom 16 in β position,
R₂ is F, R₃ is H,
R₆ is H or CH₃.

4. A compound according to claim 1 wherein
R₄ is
(F) —(Z)—Y
wherein
Z is —C(O) or —C(O)—X″, wherein X″ is O, Y is selected from
—R₇—CH(ONO₂)R₈
—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
—[(CH₂)ₒ—X]—[(CH₂)ᵣ—X]ₛ—(CH₂)_{q}—CH(ONO₂)—(CR₉R₁₀)ₜ—CH(ONO₂)R₈
wherein
R₇ is a straight or branched C₁-C₁₀ alkylene,
R₈ is H or —CH₃,
R₉ and R₁₀ at each occurrence are independently H or
—CH₃,
o and r are integers from 1 to 4,
p and s are from 1 to 4,
q is from 0 to 4,
t is 0 or 1.

5. A compound according to claim 4 wherein
R₁ is CH₃ linked to the carbon atom 16 in β position,
R₂ is F and R₃ is H.

6. A compound according to claim 1 wherein
R₄ is selected from: 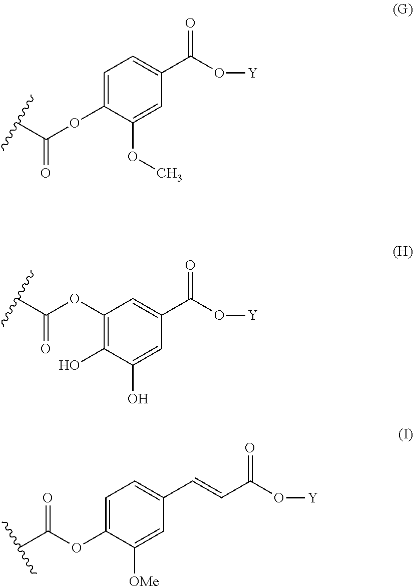
wherein
Y is selected from
—R₇—CH(ONO₂)R₈
—R₇—CH(ONO₂)—(CR₉R₁₀)ₜ—CH(ONO₂)R₈
—[(CH₂)ₒ—X]ₚ—[(CH₂)ᵣ—X]ₛ—(CH₂)_{q}—(CR₉R₁₀)ₜ—CH(ONO₂)R₈
wherein
R₇ is a straight C₁-C₁₀ alkylene;
R₈ is H or —CH₃;
R₉ and R₁₀ at each occurrence are independently H or —CH₃;
o and r are integers from 1 to 4,
p and s are from 1 to 4,
q is from 0 to 4,
t is 0 or 1,
X is O.

7. A compound according to claim 6 wherein
R₁ is CH₃ linked to the carbon atom 16 in β position,
R₂ is F and R₃ is H.

8. A compound according to claim 1 wherein
R₄ is selected from:
(A) —R¹—CH(NHR²)—C(O)—O—Y
(B) —R¹—CH(COOH)NH—C(O)—Y
(C) —R¹—CH(COOH)—O—C(O)—Y
(D) —C(O)CH(R³)—NH—C(O)—Y
(E) —C(O)CH₂—CH(R⁴)—NH—C(O)—Y
wherein
Y is selected from
—R₇—CH(ONO₂)R₈
—R₇—CH(ONO₂)—(CR₉R₁₀)ₜ—CH(ONO₂)R₈
wherein
R₇ is a straight C₁-C₁₀ alkylene;
R₈ is H or —CH₃;
R₉ and R₁₀ at each occurrence are independently H or —CH₃;
t is 0 or 1.

9. A compound according to claim 8 wherein
R^{1a} is 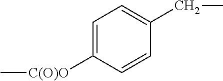
R^{1b}) is —C(O)—CH₂—,
R³ is H or —CH₃,
R⁴ is —H or —CH₃.

10. A compound according to claim 8 wherein
R₁ is CH₃ linked to the carbon atom 16 in β position,
R₂ is F and R₃ is H.

11. A compound according to claim 1 selected from the followings

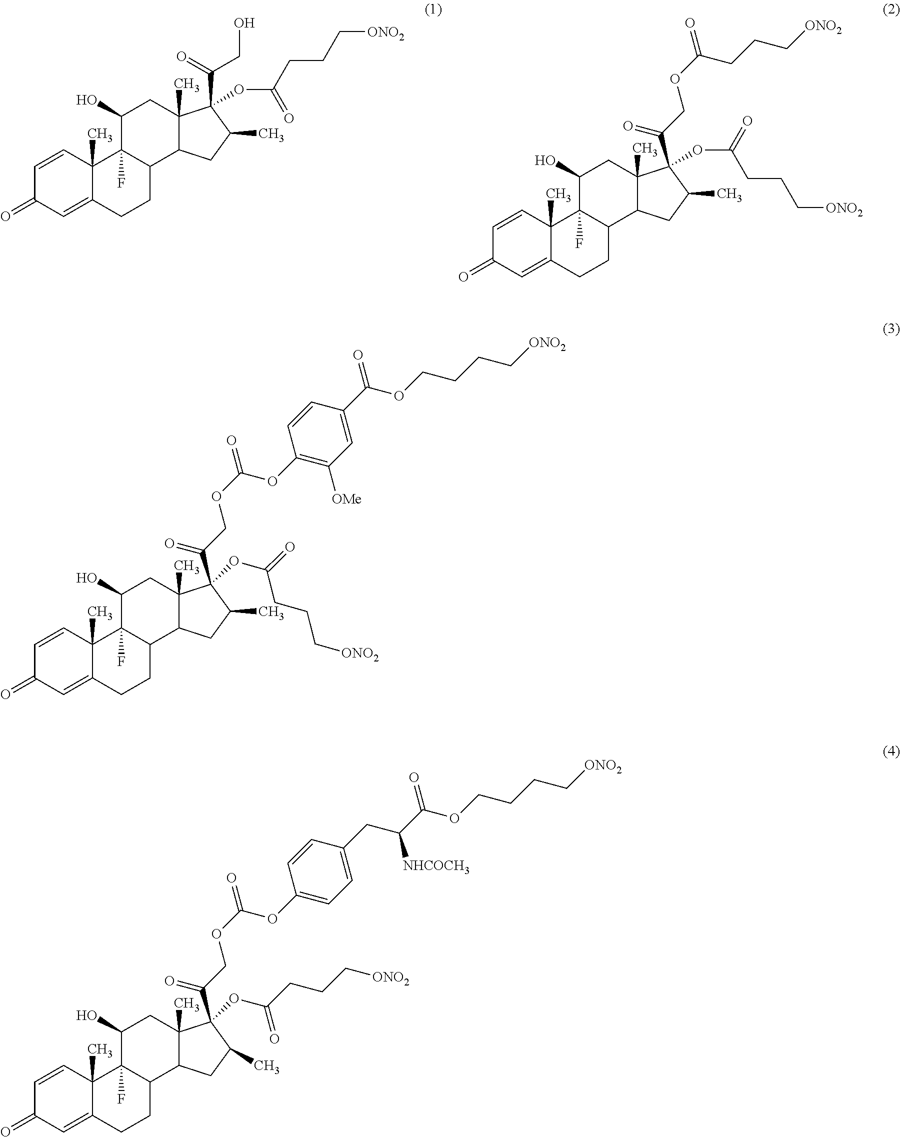

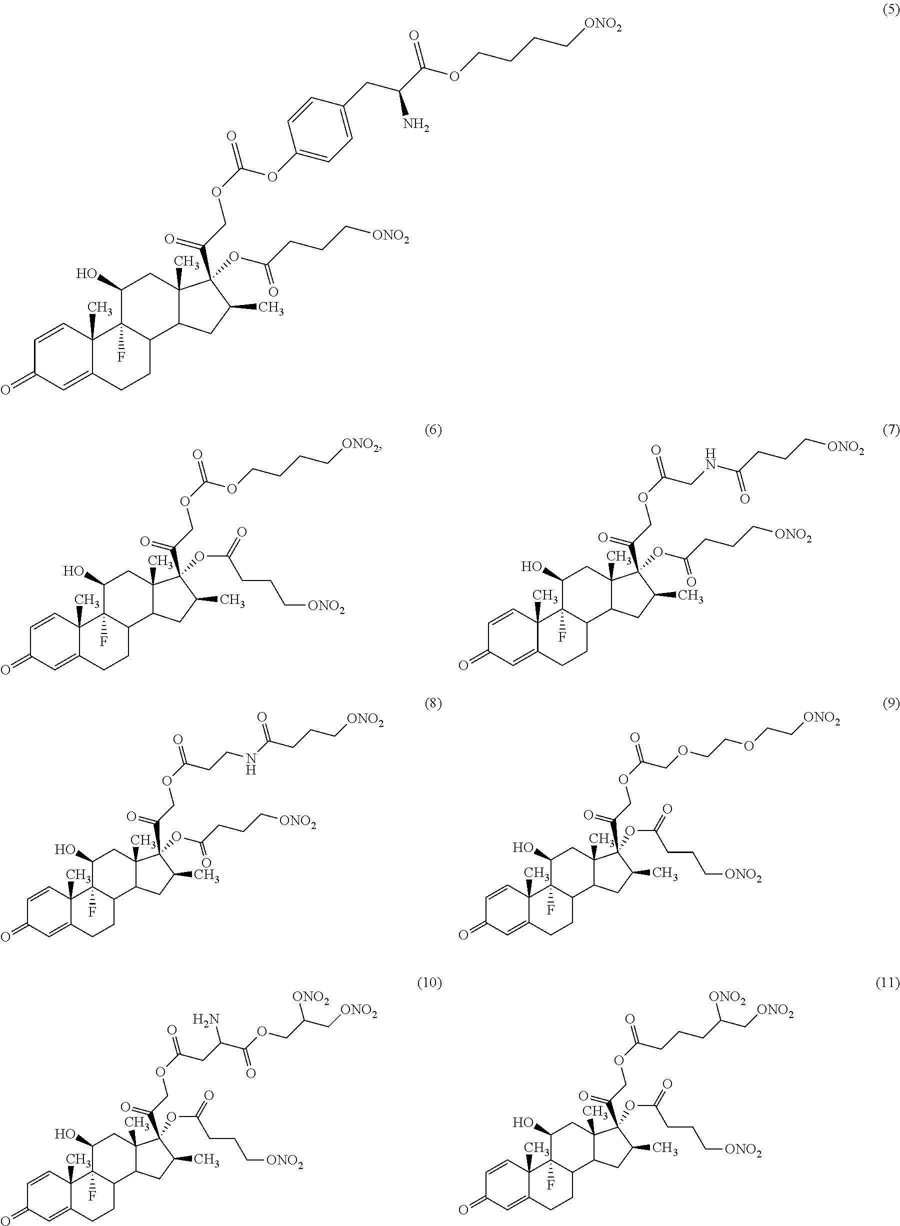

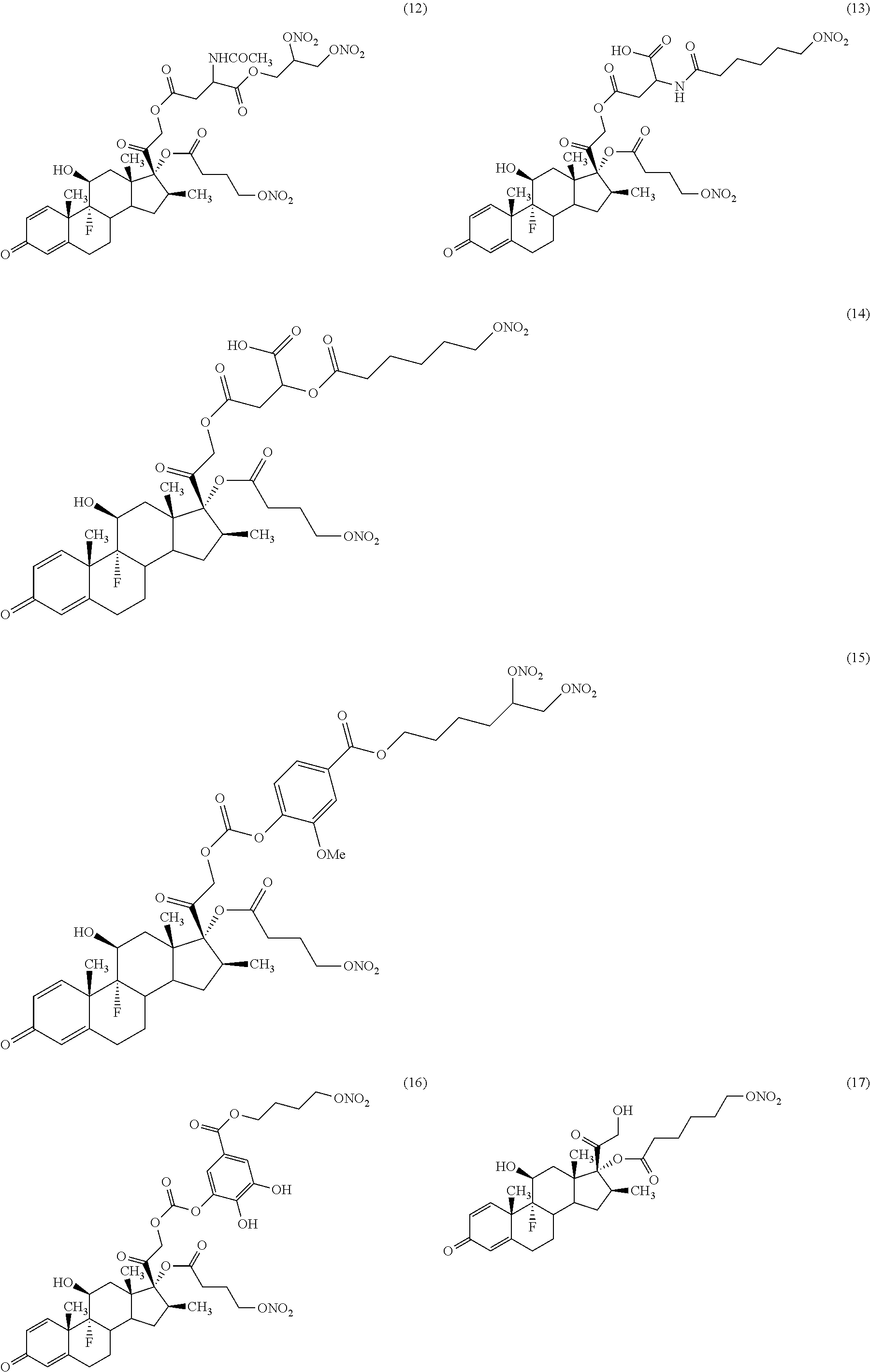

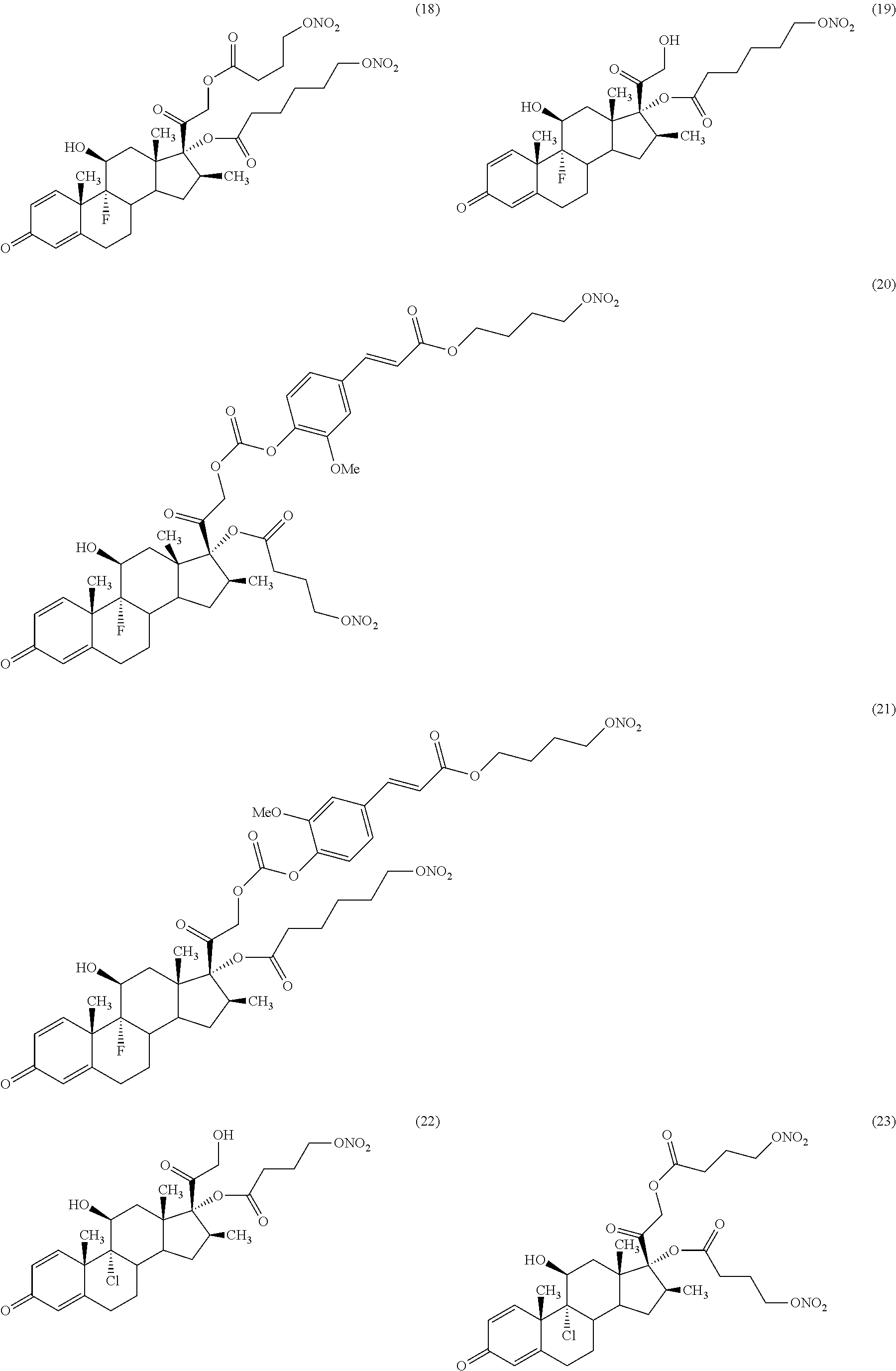

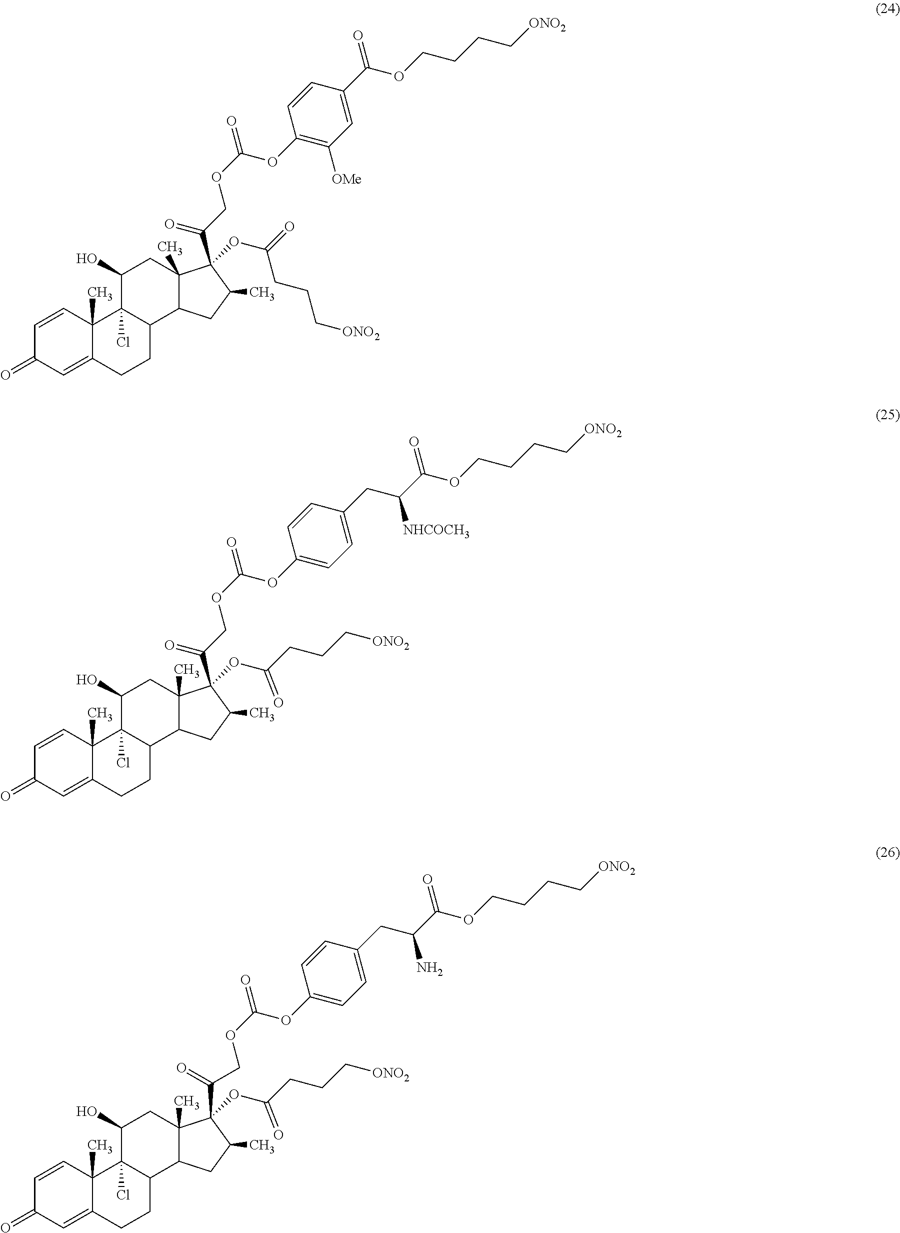

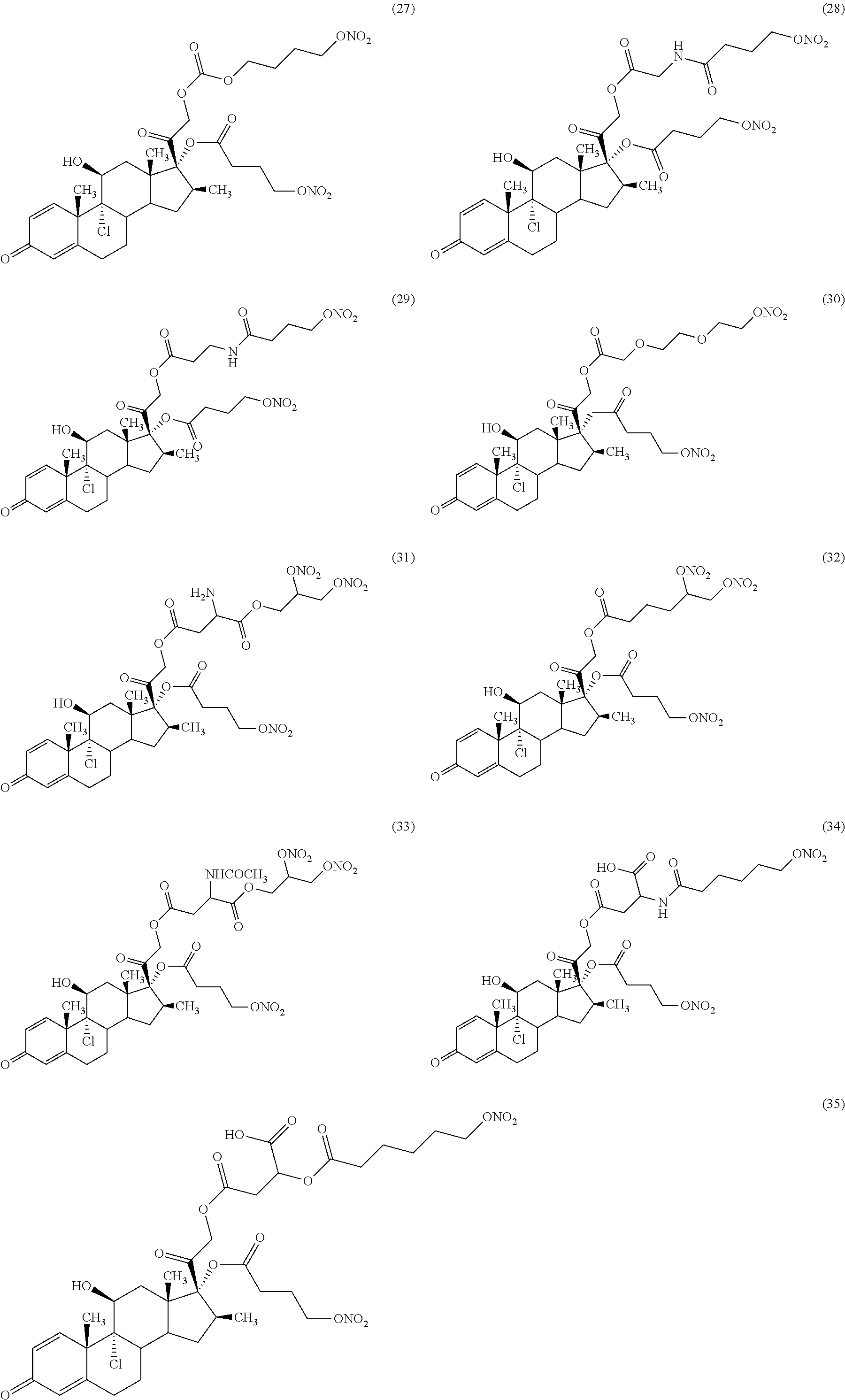

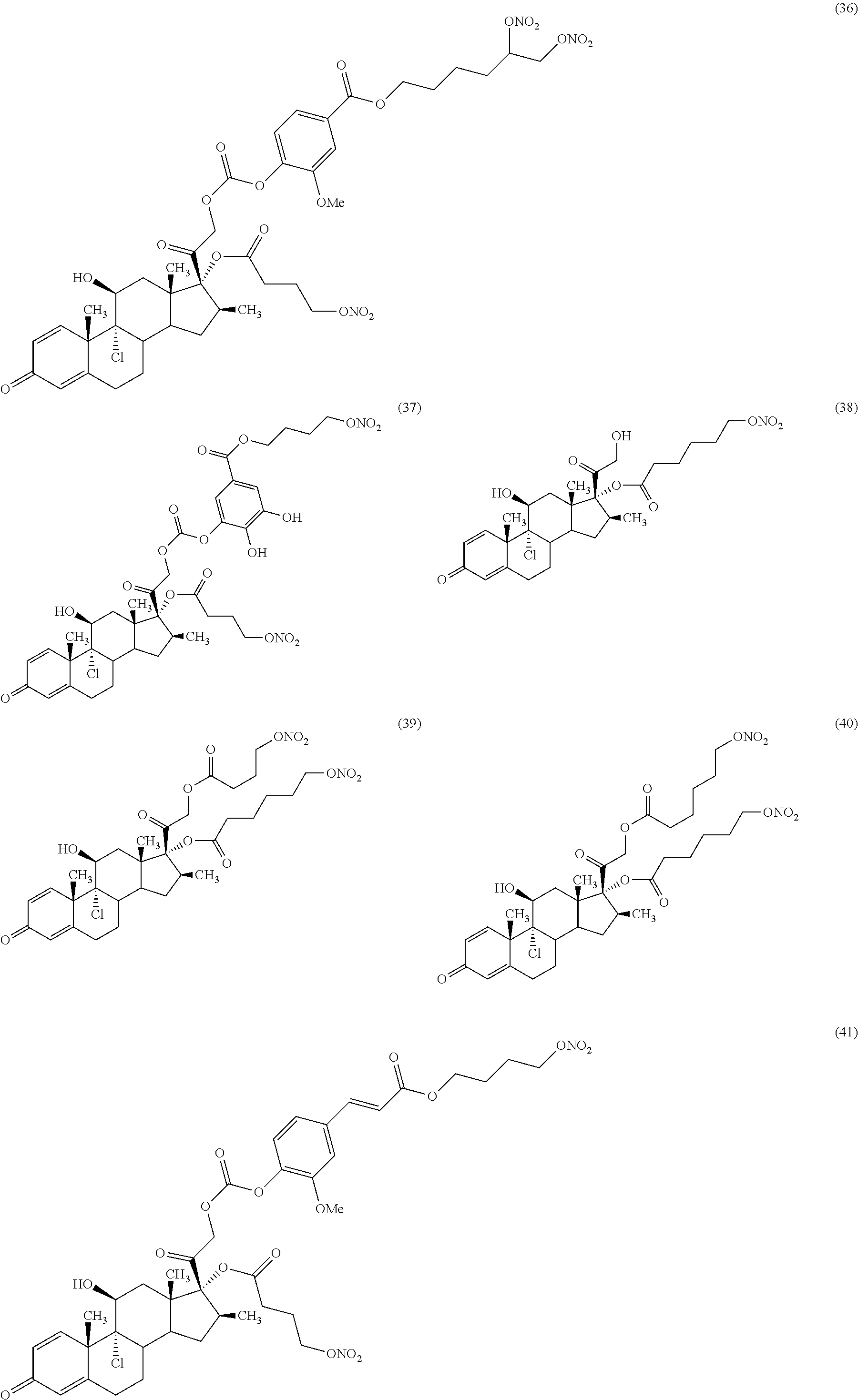

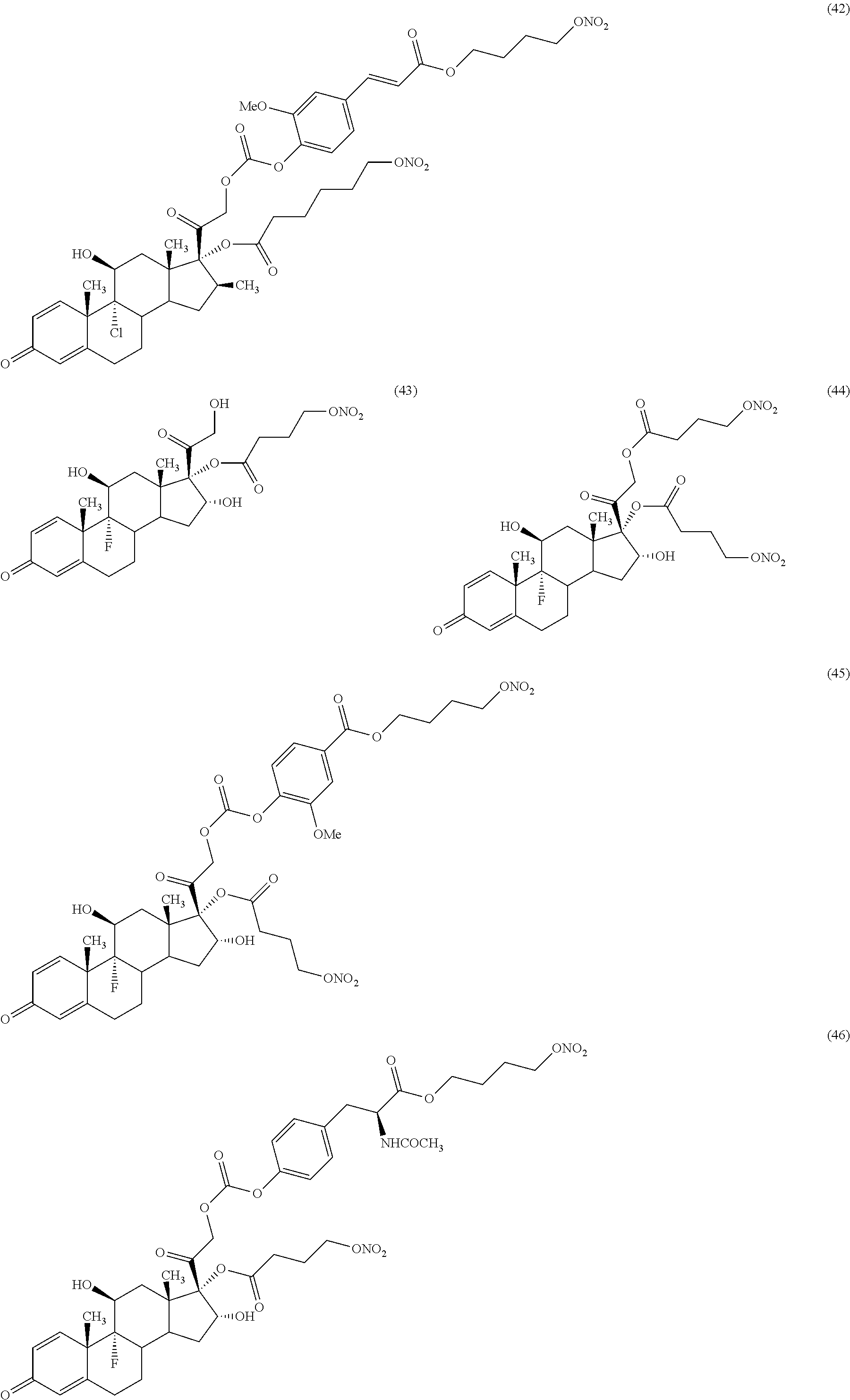

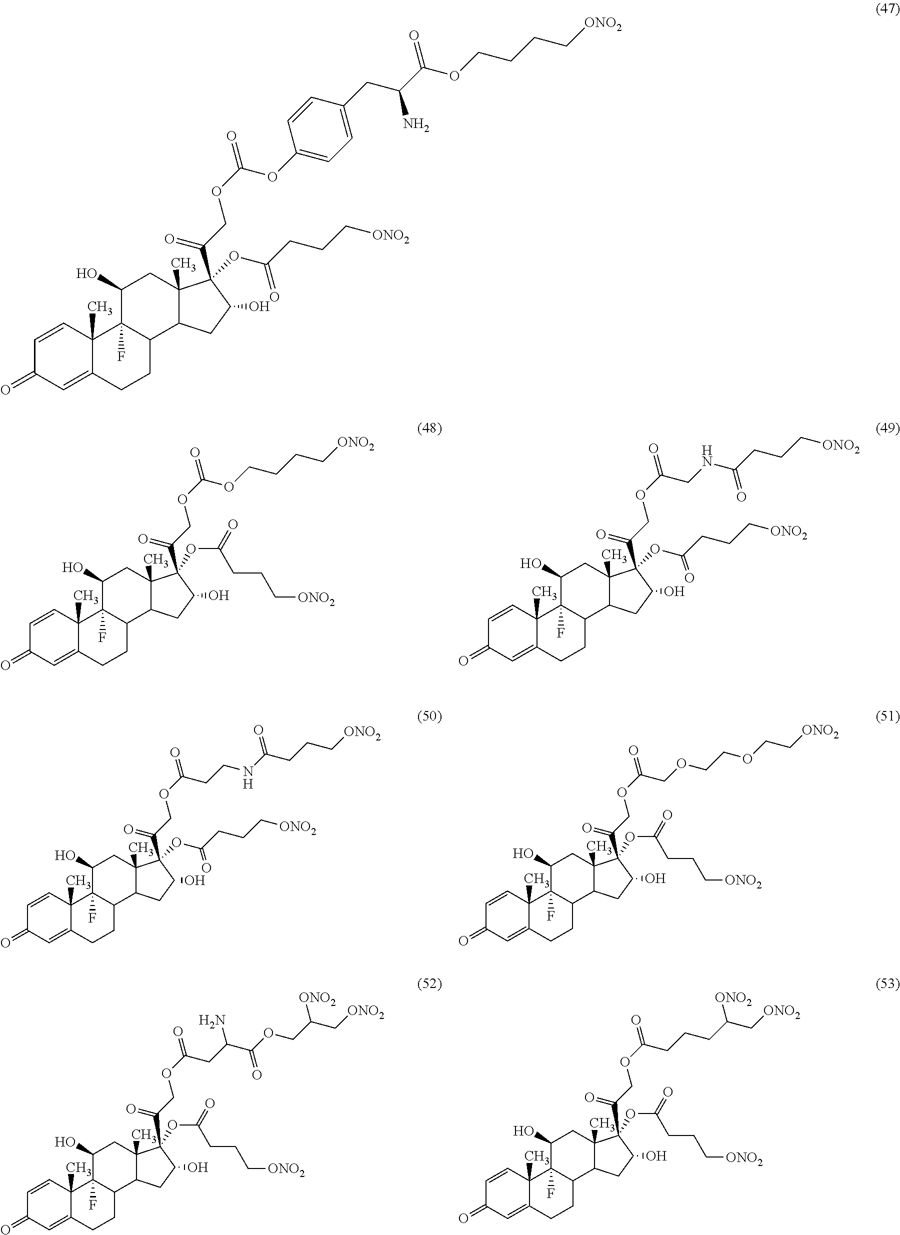

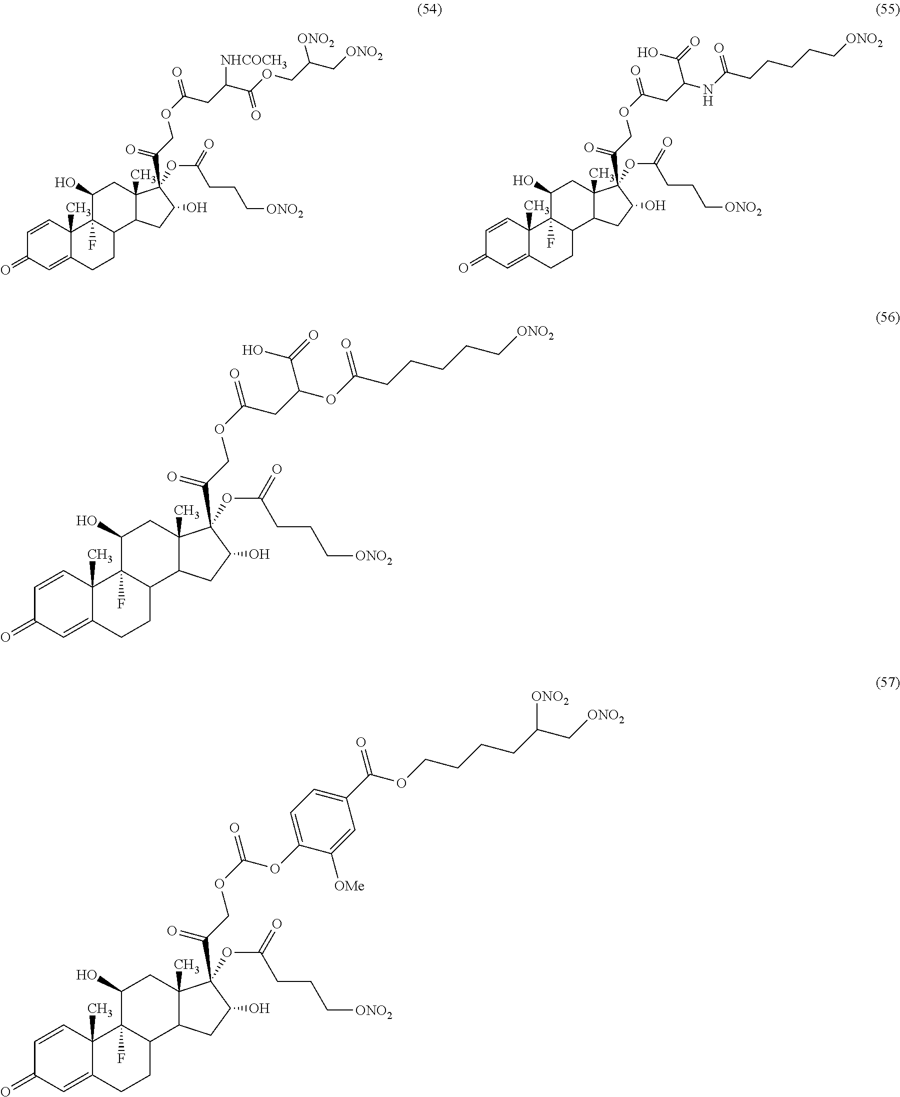

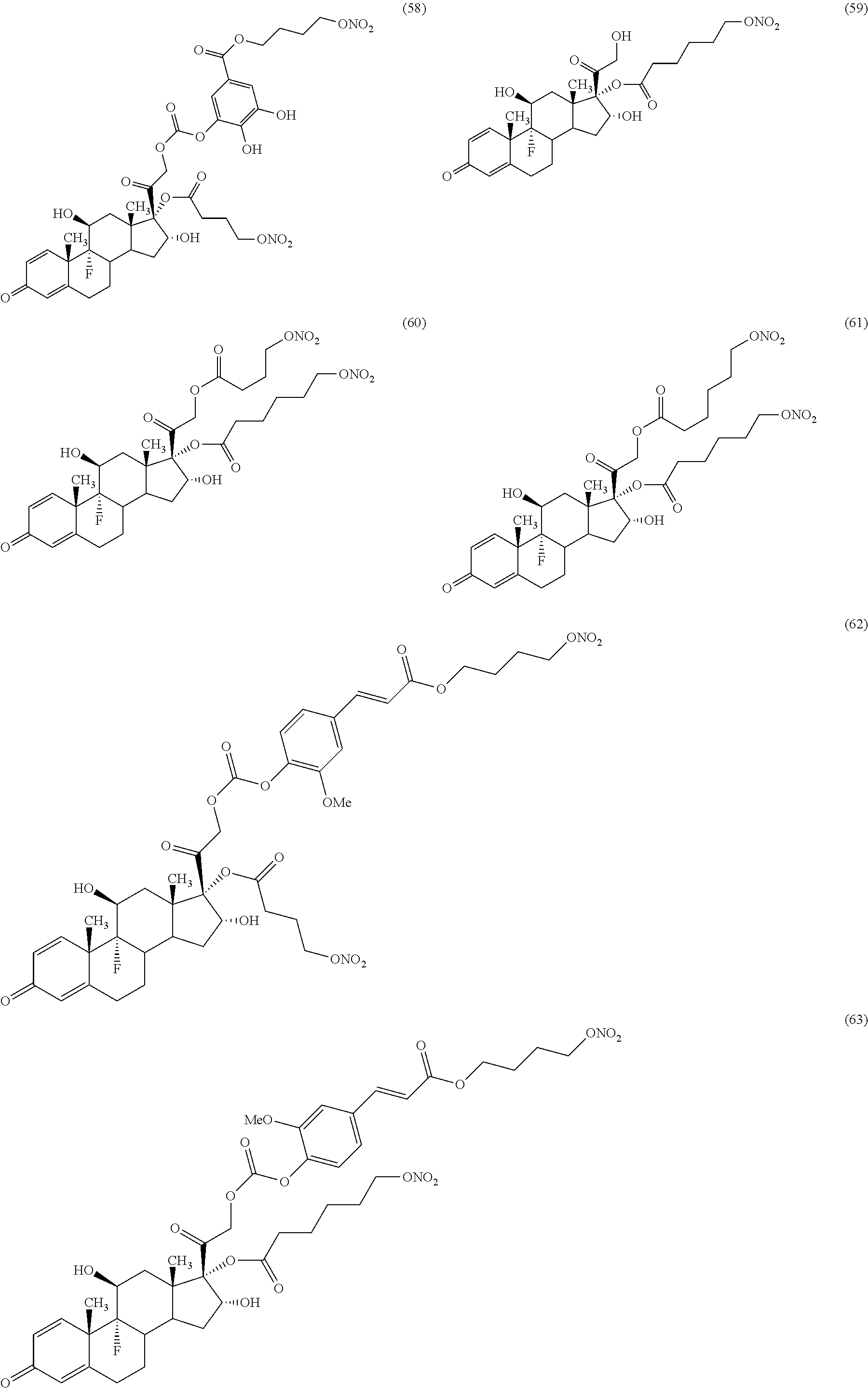

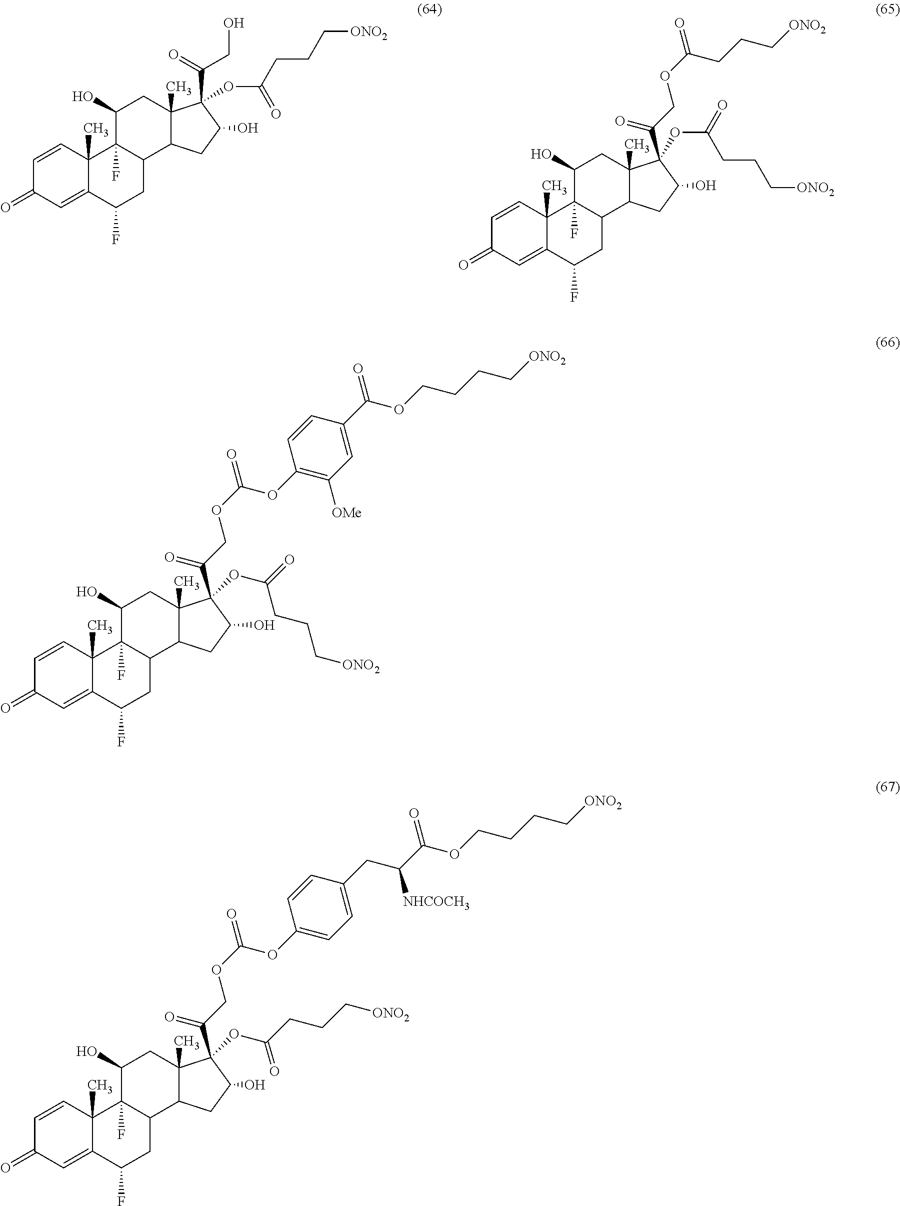

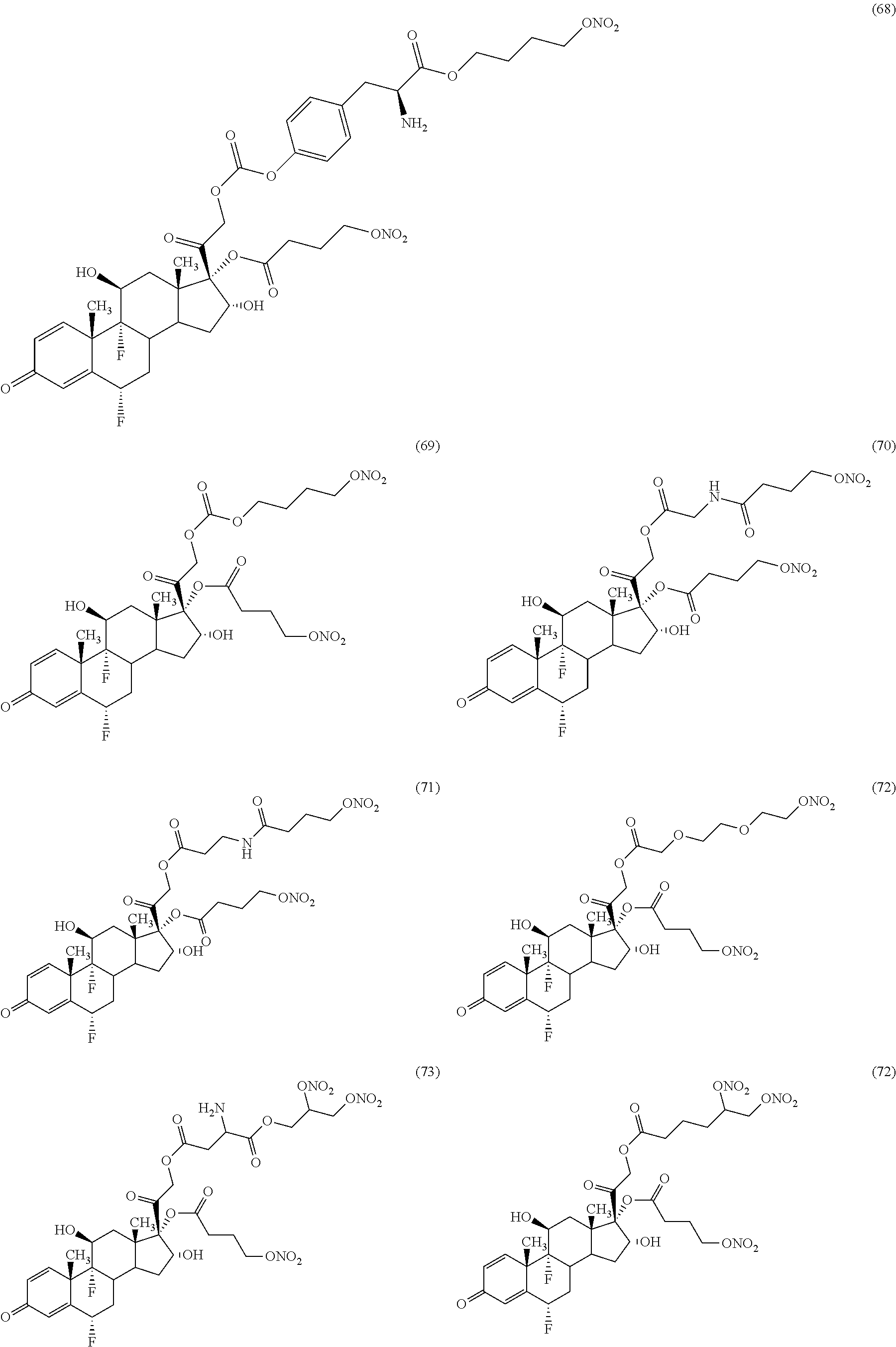

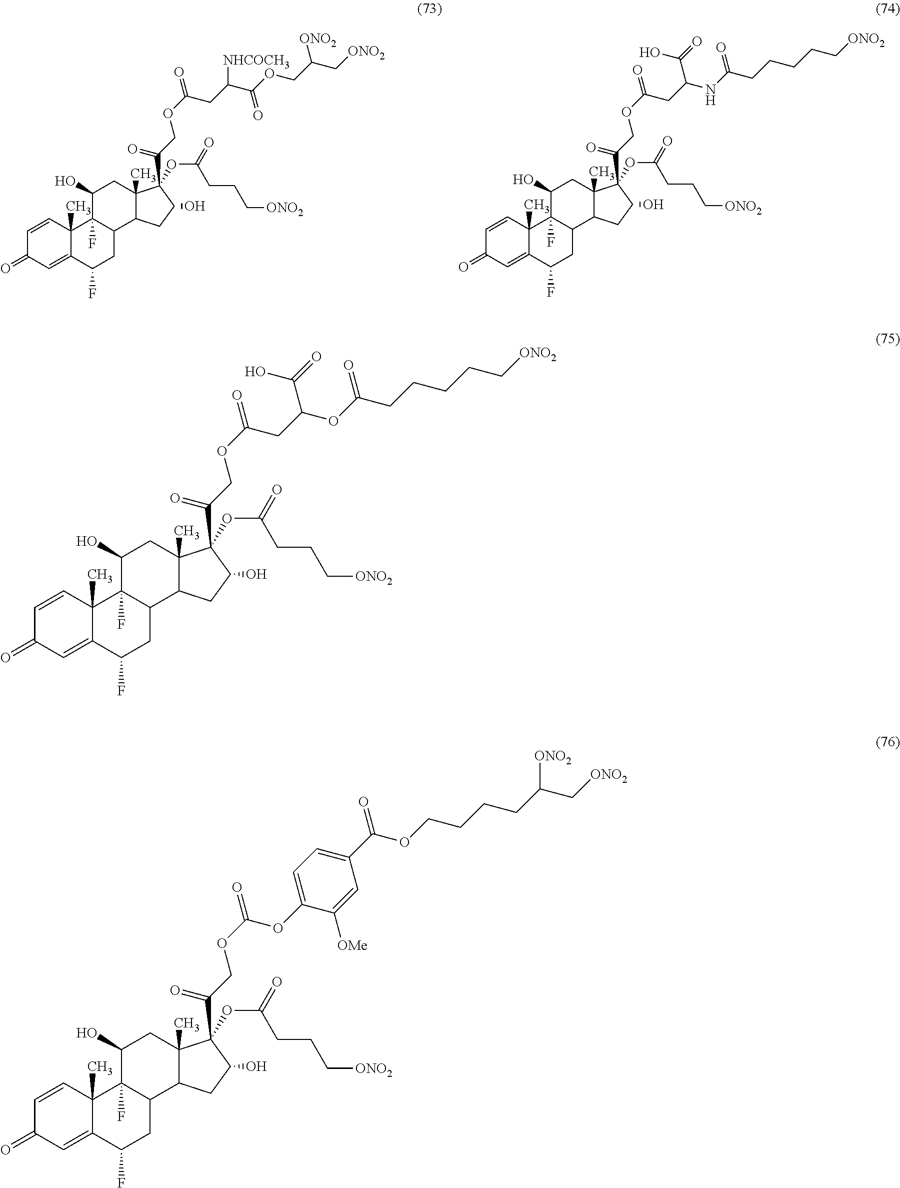

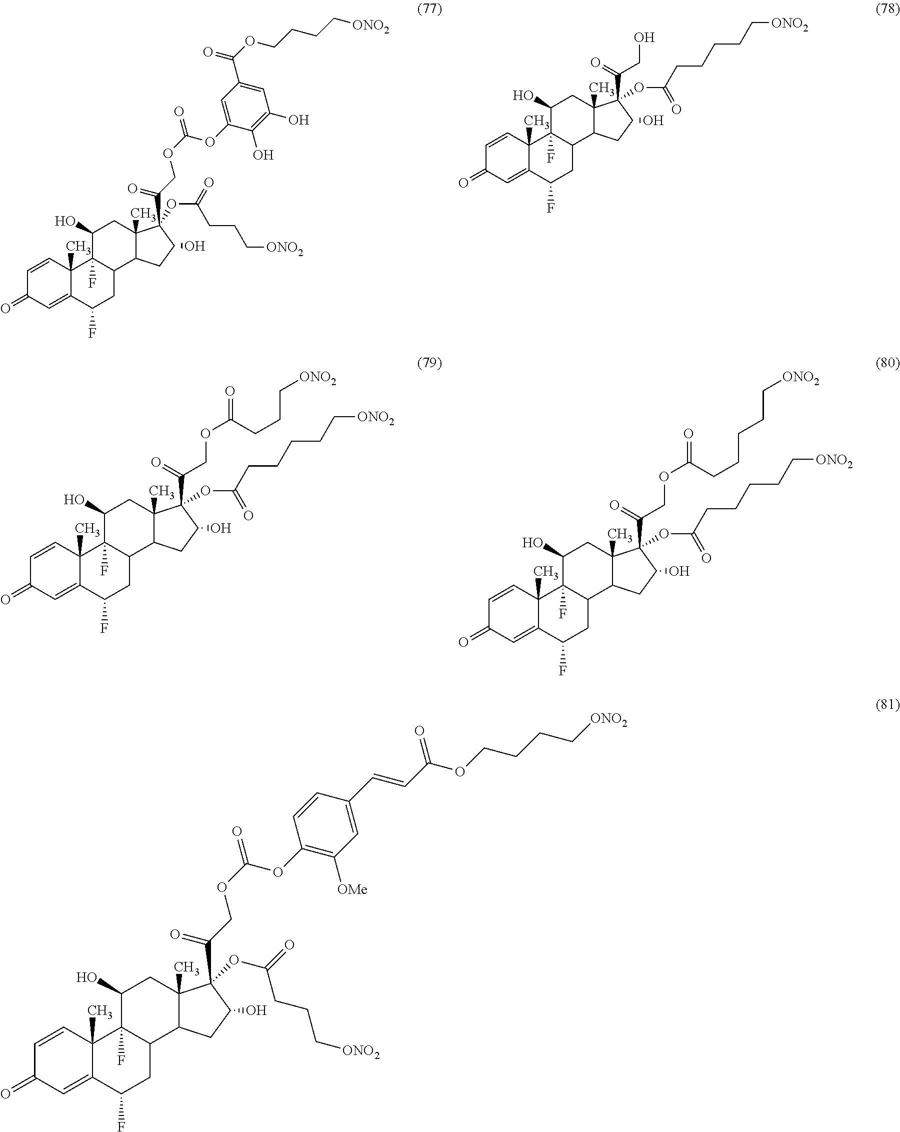

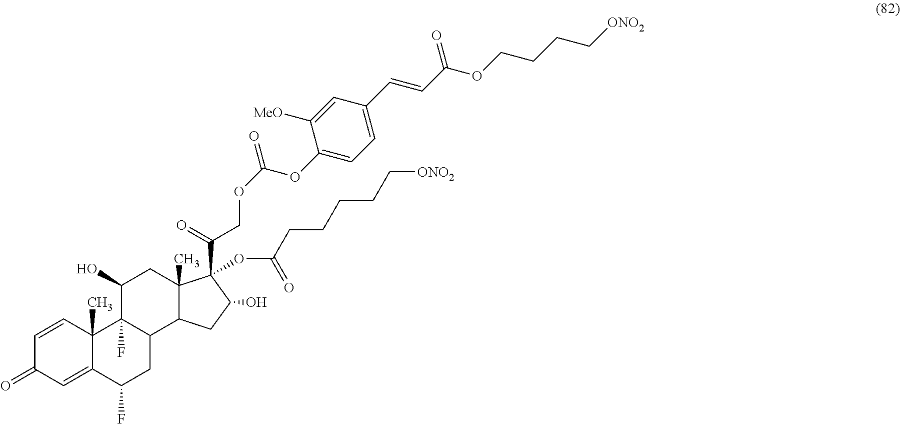

12. A compound according to claim 1 for use as medicament.

13. A compound according to claim 1 for use in the treatment of inflammatory diseases.

14. A compound according to claim 1 for use in the treatment of ocular diseases.

15. A compound according to claim 1 for use in the treatment of diabetic macular edema, diabetic retinopathy, macular degeneration, age-related macular degeneration and other diseases of retina and macula lutea.

16. A pharmaceutical composition comprising a pharmaceutically effective amount of at least a compound according to claim 1 and ophthalmically acceptable excipients in a suitable form for intravitreal or periorbital administration.

17. A pharmaceutical composition according to claim 16 for use in the treatment of inflammatory diseases.

18. A pharmaceutical composition according to claim 17 for use in the treatment of ocular diseases.

19. A pharmaceutical composition according to claim 18 for use in the treatment of diabetic macular edema, diabetic retinopathy, macular degeneration, age-related macular degeneration and other diseases of retina and macula lutea.
